(19) United States
(12) Patent Application Publication
(10) Pub. No.: US 2015/0132810 A1
(43) Pub. Date: May 14, 2015

(54) INTEGRATED PROCESSES FOR ANAEROBICALLY BIOCONVERTING HYDROGEN AND CARBON OXIDES TO OXYGENATED ORGANIC COMPOUNDS

(71) Applicant: Robert Hickey, Okemos, MI (US)

(72) Inventor: Robert Hickey, Okemos, MI (US)

(73) Assignee: Coskata, Inc., Warrenville, IL (US)

(21) Appl. No.: 14/599,908

(22) Filed: Jan. 19, 2015

Related U.S. Application Data
(63) Continuation-in-part of application No. 13/243,159, filed on Sep. 23, 2011, now Pat. No. 8,936,927.
Application No. 13/243,159 is a continuation-in-part of application No. 13/243,426, filed on Sep. 23, 2011.
Application No. 13/243,426 is a continuation-in-part of application No. 14/176,013, filed on Feb. 7, 2014.
Application No. 14/176,013 is a continuation-in-part of application No. 14/327,279, filed on Jul. 9, 2014.
Application No. 14/327,279 is a continuation-in-part of application No. 14/327,249, filed on Jul. 9, 2014.

(51) Int. Cl. C12P 7/00

(52) U.S. Cl. CPC C12P 7/00
USPC 435/132

(57) ABSTRACT

Integrated processes are provided for the bioconversion of syngas to oxygenated organic compound with the ability to recover essential compounds for the fermentation and recycle the compounds to the fermentation.

### CROSS REFERENCE TO RELATED APPLICATIONS

This application is a continuation in part of the following: U.S. application Ser. No. 13/243,159 filed Sep. 23, 2011; U.S. application Ser. No. 13/243,426 filed Sep. 23, 2011; U.S. application Ser. No. 14/176,013 filed Feb. 7, 2014; U.S. application Ser. No. 14/327,279 filed Jul. 9, 2014; and U.S. application Ser. No. 14/327,249 filed Jul. 9, 2014, the disclosure of all of which are hereby incorporated by reference in their entirety.

### BACKGROUND OF THE INVENTION

1. Field of the Invention

This invention pertains to integrated processes for anaerobically bio-converting of hydrogen and carbon oxides to oxygenated organic compounds by contact with microorganisms in a fermentation system with a high conversion efficiency of both hydrogen and carbon oxides.

2. Description of the Prior Art

Anaerobic fermentations of hydrogen and carbon monoxide involve the contact of the substrate gas in a liquid aqueous menstruum with microorganisms capable of generating oxygenated compounds such as ethanol, acetic acid, propanol and n-butanol. The production of these oxygenated organic compounds requires significant amounts of hydrogen and carbon monoxide. For instance, the theoretical equations for the conversion of carbon monoxide and hydrogen to ethanol are:

6CO+3H₂O→C₂H₅OH+4CO₂

6H₂+2CO₂→C₂H₅OH+3H₂O.

As can be seen, the conversion of carbon monoxide results in the production of oxygenated organic compound and generation of carbon dioxide. The conversion of hydrogen involves the consumption of hydrogen and carbon dioxide, and this conversion is sometimes referred to as the H₂/CO₂ conversion. For purposes herein, it is referred to as the hydrogen conversion.

Typically the substrate gas for carbon monoxide and hydrogen conversions is or is derived from a synthesis gas (syngas) from the gasification of carbonaceous materials, reforming of natural gas and/or biogas from anaerobic digestion or from off-gas streams of various industrial methods. The gas substrate contains carbon monoxide, hydrogen, and carbon dioxide and usually contains other components such as water vapor, nitrogen, methane, ammonia, hydrogen sulfide and the like. (For purposes herein, all gas compositions are reported on a dry basis unless otherwise stated or clear from the context.)

Certain low cost waste materials can make the syngas conversion processes more economically attractive. Such waste material include the organic fraction of municipal solid waste [OFMSW], biological waste sludge, source separated organics, or green wastes (i.e. food waste (FW) from supermarkets, cafeterias, etc.), high strength food processing wastewaters (whey, whey permeate, vegetable preparation WW, etc.,), distillery byproducts including stillage and thin stillage and others. Although these sources are abundant they can be problematic to use as feedstocks for gasification and subsequent fermentation to ethanol (and other liquid products). This is due to their high moisture content and/or the presence of compounds in the waste that make them hard to manage or produce undesirable compounds in the product Syngas. These wastes can be valuable sources of nutrients (such as nitrogen, sulfur and phosphorus), as well as CH₄ and CO₂, all useful for fermentation of Syngas to chemicals processes.

In the case of biological waste sludge this can include spent microorganisms from the fermentation itself. Even for relatively large Syngas to Alcohol (or other chemicals) production facilities these wastes can still represent a significant fraction of these needed resources for the Syngas fermentation process. The proper integration of the anaerobic processing i.e. anaerobic digestion (AD), post treatment and use of the fractionated components can represent a considerable savings in overall production costs and result in an overall process that is much more sustainable.

Heretofore, the use of high moisture, renewable feedstocks in such area has been frowned upon due to due to their high moisture content and/or the presence of compounds in the waste that make them difficult to manage or which produce undesirable compounds in the Syngas feedstream. One aspect of the instant invention overcomes these issues and secures even greater value from these materials.

Fermentation processes may be made more attractive from capital and energy costs standpoints by avoiding costs to procure nutrients such as sulfur. Microorganisms used in metabolic processes require nutrients and micronutrients. One of the required nutrients is a source of reduced sulfur, usually in the form of a sulfide such as cysteine. Sulfur is a key nutritional need of anaerobic microorganisms used in these fermentations to produce oxygenated organic compound. Any interruptions in the supply of sulfur nutrient results in an almost immediate decrease in the rate and stability of the fermentation.

Organic sulfur sources, such as cysteine are expensive, and alternative sources of sulfur to meet this nutritional need have been sought. Less expensive sources of sulfur include, but are not limited to, hydrogen sulfide, and sulfite, bisulfite, thiosulfate and metabisulfite anions. However, typical aqueous menstruum for the bioconversion of carbon monoxide and of hydrogen and carbon dioxide are acidic. Consequently the equilibrium for hydrogen sulfide, which provides the sulfhydryl anion that is believed to be used by the microorganisms, strongly favors gaseous hydrogen sulfide as opposed to the sulfhydryl anion, and gaseous hydrogen sulfide rapidly exits the aqueous menstruum. Sulfite, bisulfite, thiosulfate and metabisulfite are rapidly transformed to sulfide leaving virtually no residual sulfite, bisulfite, thiosulfate or metabisulfite anion in the aqueous menstruum. The sulfide, which will primarily exist as hydrogen sulfide, thus will rapidly evolve from the aqueous menstruum into the gas phase.

Although hydrogen sulfide is less expensive than, say, cysteine, it is toxic and thus requires special handling and is particularly dangerous in pure form. Accordingly, if hydrogen sulfide is to be a viable source of reduced sulfur, generation at the site of the fermentation at the rate required to avoid significant storage of hydrogen sulfide would be desired.

Numerous processes exist that generate hydrogen sulfide, either as the sought product or as a contaminant in another process. For instance, Velt, et al., in U.S. Patent Publication No. 2010/0221804 propose an integrated ethanol and biogas system where thin stillage is processed to generate a biogas. At paragraph 0028, the aforemention publication states that the biogas contains methane and carbon dioxide and can also include hydrogen, hydrogen sulfide and ammonia and suggests that the biogas can be used for heating or operating various types of engines to produce mechanical or electrical power. Although hydrogen sulfide can be recovered from gas streams, processes for the recovery necessarily incur capital and operating costs. These costs thus reduce the attractiveness of these hydrogen sulfide-containing gas streams being a source of sulfur for fermentation processes.

Offerman, in U.S. Published Patent Application No. 2008/0220489 discloses a process in which biogas is generated from wastes, such as manure, and the biogas is converted to syngas for synthesis of liquid fuels. The applicant discusses the use of Fe⁺² from iron-reducing microorganisms in the fermentation to generate the biogas or amine-containing resin to reduce the concentration of hydrogen sulfide in the biogas, and thus in the ultimate fuel product.

Balmat, in U.S. Pat. No. 4,200,523 discloses processes for removing sulfate ions from dilute aqueous streams by contact with Desulfovibrio sulfate-reducing bacteria to convert the sulfate to sulfide and removing the sulfide ions. The use of an electron donor (gaseous hydrogen) is required.

Another issue is disruption in the supply of sulfur nutrients. Even dosing the aqueous menstruum with sulfite, bisulfite, thiosulfate or metabisulfite anion at levels well in excess of the cell sulfur requirements may not provide the needed sulfur nutrient required to maintain the population of microorganisms in such event. And such overdosing results in the tail gas from the anaerobic bioconversion process having significant concentrations of hydrogen sulfide. Thus, in addition to increasing the cost of supplying sulfur nutrient, accommodations may be required to remove or reduce the concentration of sulfur compounds in the tail gas to enable the use or disposal of the tail gas.

Another difficulty syngas fermentation processes suffer from the poor solubility of the gas substrate, i.e., carbon monoxide and hydrogen, in the liquid phase of the aqueous menstruum. Munasinghe, et al., in Biomass-derived Syngas Fermentation in Biofuels: Opportunities and Challenges, Biosource Technology, 101 (2010) 5013-5022, summarize volumetric mass transfer coefficients to fermentation media reported in the literature for syngas and carbon monoxide in various reactor configurations and hydrodynamic conditions. A number of conditions can enhance the mass transfer of syngas to the liquid phase. Increasing the interfacial area between the gas feed and the liquid phase can improve mass transfer rates. For stirred tank reactors (CSTRs), increasing the agitation of the impeller improves mass transfer as smaller bubble sizes are obtained. The authors report that in one study, the mass transfer obtained for a bubble column reactor (also known as a bubble column bioreactor—BCBR) was higher than that for a stirred tank reactor mainly due to the higher interfacial surface area obtained by using a microbubble sparger with the bubble column reactor. They report the findings of another study where it was concluded that the axial mixing of microbubble dispersions in bubble column reactors was considerably less than that of the conventional bubble column reactors. Munasignhe, et al., in a later published paper, Syngas Fermenation to Biofuels Evaluation of Carbon Monoxide Mass Transfer Coefficent (k_{L}a) in Different Reactor Configurations, Biotechol. Prog., 2010, Vol. 26, No. 6, pp 1616-1621, combine a sparger (0.5 millimeter diameter pores) with mechanical mixing at various rotational rates to provide enhanced mass transfer. They also report on prior work by others who used a stirred tank and microbubble sparger to obtain high volumetric mass transfer coefficients.

For a syngas to oxygenated organic compound fermentation process to be commercially viable, capital and operating costs must be sufficiently low that it is at least competitive with alternative biomass to oxygenated organic compound processes. For instance, ethanol is commercially produced from corn in facilities having name plate capacities of over 100 million gallons per year. Accordingly, the syngas to oxygenated organic compound fermentation process must be able to take advantage of similar economies of scale. Thus, a commercial scale facility may require at least 20 million liters of fermentation reactor capacity. Problems with stirred tank reactors are capital costs, the significant amount of energy needed for gas transfer and mixing, and the need for plural stages to achieve high conversion of gaseous substrates, thus stirred tank reactors face considerable difficulties in being justified for these commercial-scale facilities. Reported by Munasignhe, et al., other syngas fermentation reactor types such a bubble column reactors and air lift (jet loop) reactors are less costly to manufacture and operate yet can provide good mass transfer rates of syngas to the liquid phase. However, microbubble spargers, especially for very small microbubbles, use significant amounts of energy and are prone to fouling. Accordingly, other means for generating microbubbles such as injectors using a motive fluid that are not prone to fouling, are preferred. Co-pending U.S. Pat. No. 8,795,995, discloses the use of injectors to supply gas feed to an anaerobic fermentation in a deep reactor to make a liquid product such as ethanol wherein the presence of the liquid product enables the injector to produce a dispersion of microbubbles.

Continuous syngas fermentation processes typically produce co-produced oxygenated organic compounds in addition to the sought, product oxygenated organic compound. The co-produced oxygenated organic compounds can be co-metabolites that are not desired or intermediate metabolites in the bioproduction of the sought, product oxygenated organic compound. Also, co-produced oxygenated organic compounds can be produced by contaminating, or adventitious, microorganisms present in the aqueous fermentation broth. In some instances, these co-produced oxygenated organic compounds may be produced at rates, relative to the production rate of the sought, product oxygenated organic compound, that cause a build-up of the co-produced oxygenated organic compound in the aqueous broth. This build-up of the co-produced oxygenated organic compound is particularly untoward where the co-produced oxygenated organic compound reaches concentration levels that are inhibitory or toxic to the microorganisms used for the syngas fermentation. In some other instances, the co-produced oxygenated organic compound, when at sufficient concentrations, can adversely affect the metabolic pathways of certain microorganisms used for the bioconversion of syngas. For instance, where an alcohol is the sought, product oxygenated organic compound, with some microorganisms, the presence of certain concentrations of free carboxylic acids can induce a product distribution shift in which the microorganisms to generate a higher percentage of carboxylic acids. The exponentially increasing production of the acids leads to an increasing acidity in the fermentation broth causing an eventual loss of the microorganism being able to maintain cell membrane potential and loss of the population of microorganisms.

Although the fermentation broth could be discarded in the event that the concentration of the undesired organic compound becomes excessive, nutrients for the fermentation would also be lost. Additionally, for commercial-scale bioreactors, disposal of the large volume of aqueous broth in a bioreactor can be problematic depending upon the capacity of the waste water treatment system. Since a commercial-scale bioreactor may contain in excess of 1 million liters of aqueous broth, it is likely that the waste water from the bioreactor would have to be slowly discharged to the waste water treatment system to prevent exceeding capacity. Thus, the downtime of the affected bioreactor would be extended, resulting in a further loss of production. The amount of water lost could also be an economic loss.

In some instances, the undesired organic compound may be capable of being selectively removed such as by ion-exchange resins or membrane separations. These approaches may not provide suitable selectivity and are capital intensive yet may only be required sporadically or intermittently. But when needed, these unit operations must be able treat large quantities of fermentation broth in a short period of time. Moreover, they suffer from potential issues with fouling.

Accordingly, effective processes for converting syngas to Liquid Products need to have the capability of removing at least one undesired organic compound from an anaerobic fermentation broth that involve low capital expense yet can relatively quickly effect the reduction in concentration of the at least one undesired organic compound while retaining the fermentation broth anaerobic and retaining nutrients. Such desired processes should be capable of treating the large volumes of fermentation broth associated with commercial-scale bioreactors even on a sporadic or intermittent basis.

In addition to economies of scale, the processes need to obtain high conversion efficiencies of the syngas to oxygenated organic compounds. Syngas and other carbon monoxide and hydrogen-containing gas feeds are typically more expensive than equivalent heat content amounts of fossil fuels. Hence, a desire exists to use these gases effectively to make higher value products. The financial viability of any conversion process, especially to commodity chemicals such as ethanol and acetic acid, will be dependent upon the efficiency of conversion of the carbon monoxide and hydrogen, the selectivity of conversion to the sought products and the energy costs to effect the conversion.

Accordingly, a multitude of challenges are faced when seeking to take advantage of the benefits of stirred tank reactors for the conversion of syngas to oxygenated organic compound at the large scale required for commercial viability. In their review article, Munasignhe, et al., report that the mass transfer coefficient for slightly soluble gaseous substrates is dependent upon the difference in partial pressures in the gas and in the liquid phases. The authors state at page 5017:
“High pressure operation improves the solubility of the gas in the aqueous phase. However, at higher concentrations of gaseous substrates, especially CO, anaerobic microorganisms are inhibited.”

Other workers have understood that the presence of excess carbon monoxide can adversely affect the microorganisms and their performance. See paragraphs 0075 through 0077 and 0085 though 0086 of US Published Patent Application No. 20030211585 (Gaddy, et al.) disclosing a continuously stirred tank bioreactor for the production of ethanol from microbial fermentation. At paragraph 0077, Gaddy, et al., state:
“The presence of excess CO unfortunately also results in poor H₂ conversion, which may not be economically favorable. The consequence of extended operation under substrate inhibition is poor H₂ uptake. This eventually causes cell lysis and necessary restarting of the reactor. Where this method has an unintended result of CO substrate inhibition (the presence of too much CO for the available cells) during the initial growth of the culture or thereafter, the gas feed rate and/or agitation rate is reduced until the substrate inhibition is relieved.”

At paragraph 0085, Gaddy, et al., discuss supplying excess carbon monoxide and hydrogen. They state:
“A slight excess of CO and H₂ is achieved by attaining steady operation and then gradually increasing the gas feed rate and/or agitation rate (10% or less increments) until the CO and H₂ conversions just start to decline.”

Thus a commercial-scale process using a fermentation vessel such as a deep tank fermenter such as a BCBR or CSTR must be able to balance obtaining desirable rates of diffusion of carbon monoxide into the aqueous menstruum with avoiding carbon monoxide inhibition.

Accordingly, processes are sought to take advantages of using low cost feed materials, sulfur sources recaptured from the fermentation process, effective utilization of sulfur sources, avoidance of tail gas, economical operation of a stirred tank reactor and/or without undue capital and operating costs while achieving both high conversion of gas substrate and selectivity to oxygenated organic compound. Particularly useful forms of such processes will also enhance the economics of syngas fermentation to produce oxygenated organic compound where reduced sulfur nutrient can be effectively and inexpensively supplied by the processes at an as needed rate and will have the capability to reduce or eliminate the deleterious effects of contaminating organisms.

### SUMMARY OF THE INVENTION

This invention provides robust processes for converting syngas to Liquid Products in a manner that overcomes the most significant operational challenges plaguing the efficient use of the anaerobic fermentations required for achieving commercial success of such methods.

In one aspect of this invention the provided processes can economically and effectively remove one or more co-produced oxygenated organic compounds (referred to herein individually and collectively as the Adverse Component) from an anaerobic, aqueous fermentation broth used for the bioconversion of syngas to product oxygenated organic compound. By this aspect the processes of this invention can maintain the aqueous fermentation broth as an anaerobic medium and avoid discarding the entire volume of aqueous fermentation broth. Thus, loss of downtime, and lost production of product oxygenated organic compound, can be attenuated. Moreover, by the use of this aspect the processes of this invention do not require the loss or removal of a significant mass of nutrients from the broth. The processes of this invention that include this aspect are applicable both to addressing catastrophic failures, i.e., where the population of microorganisms in the aqueous fermentation broth has been decimated by the presence of the Adverse Component, and avoidance of catastrophic failure by removal of the Adverse Component prior to reaching untoward concentrations. The Adverse Component is removed by degradation, and the primary degradation products are gaseous and can therefore be readily eliminated from the broth being treated.

In this particular aspect the processes in accordance with this invention can involve the addition of nitrate anion to the fermentation broth and contacting the broth with denitrifying microorganisms in separate vessel. Denitrification occurs under anoxic conditions whereby the nitrate anion is reduced and oxygenated organic compounds (which include the Adverse Component and any product oxygenated organic compound remaining in the broth) are oxidized to carbon dioxide. The nitrate anion is reduced to at least one reduced nitrogen compound, i.e., when the valence of the nitrogen atom is reduced, such as molecular nitrogen, nitrous oxide, nitric oxide, ammonium cation and nitrite anion. In most instances, the reduced nitrogen compound is one or more of molecular nitrogen, nitric oxide and nitrous oxide.

With this aspect of the processes of this invention the Adverse Component a fermentation operation can be practiced without additional undue capital expense. Moreover, the metabolic conditions can be the same or similar to those used for the syngas bioconversion thereby being attractive from an operating expense standpoint. Further, when this aspect of the invention is used processes do not necessarily pose the same type of fouling problems associated with the use of membranes or ion exchange resins to remove the Adverse Component. This aspect of the invention can address an unplanned event or can be used on a continuous or intermittent basis to remove Adverse Component co-produced with the product oxygenated organic compound.

In more complete terms, the aspect of the processes of this invention for removing at least one co-produced oxygenated organic compound from an anaerobic, aqueous fermentation broth used for bioconverting syngas to product oxygenated organic compound comprises supplying to the fermentation broth nitrate anion to provide a nitrate-containing broth and contacting the nitrate-containing broth with denitrifying microorganisms under anoxic bioconversion conditions to metabolically produce carbon dioxide and reduced nitrogen compound and an anaerobic fermentation broth having a reduced concentration of the at least one co-produced oxygenated organic compound. In suitable aspects at least a portion, preferably at least about 75, and sometimes at least about 90 or 95, mass percent of the product oxygenated organic compound is removed from the fermentation broth prior to supplying nitrate anion to the fermentation broth (i.e., the implementation of the denitrifying process). This aspect of the invention also pertains to continuous processes for the anaerobic bioconversion of a gas substrate comprising carbon monoxide, hydrogen and carbon dioxide in an aqueous broth containing microorganisms suitable for converting the substrate to product oxygenated organic compound, which processes comprise continuously contacting the gas substrate with the aqueous broth under acidic, anaerobic fermentation conditions to bioconvert gas substrate to oxygenated organic compound and provide a product oxygenated organic compound-containing broth and a depleted gas phase, the anaerobic fermentation conditions also producing an co-produced oxygenated organic compound and continuously withdrawing the depleted gas phase from the aqueous broth. This aspect of the invention may continuously or intermittently withdraw a portion of the broth for recovery of the product oxygenated organic compound, the withdrawal being sufficient to maintain the product oxygenated organic compound in the broth below a concentration that unduly adversely affects the microorganisms. This aspect may also continuously separate at least one product oxygenated organic compound from the withdrawn portion of the broth to provide at least one fraction rich in the at least one product oxygenated organic compound and a depleted aqueous fraction containing the at least one co-produced oxygenated organic compound while continuously or intermittently adding nitrate anion to at least a portion of the depleted aqueous fraction and thereby provide a nitrate-containing broth that contacts the nitrate-containing broth with denitrifying microorganisms under anoxic bioconversion conditions to metabolically produce carbon dioxide and reduced nitrogen compound and a treated broth having a reduced concentration of the at least one co-produced oxygenated organic compound. At least a portion of the treated broth provides an anaerobic aqueous broth for the bioconversion of gas substrate to product oxygenated organic compound.

To achieve the maximum value from use of high moisture renewable materials they must first be processed in an anaerobic treatment system to produce useable compounds which must next be segregated, and then subsequently integrated into the Syngas fermentation processes of this invention. Some such waste streams may also require some form of pretreatment before they can be anaerobically digested, such as removal of contaminating materials, size reduction, etc. By this aspect of the invention it has been found that many such waste streams can be treated anaerobically in methanogenic systems without pretreatment to produce a biogas containing primarily CH₄ and CO₂, typically in amounts of from 50 to 75% and 25 to 50%, respectively, as well as H₂S in a quantity of up to 3%. The digestion process also converts nitrogen and phosphorus in the waste streams into forms which are recoverable and useable in the Syngas fermentation process. Further processing of the digestate can be used to recover valuable trace metals and minerals for reuse.

The proper integration of anaerobic processing, post-treatment, and use of fractionated components will represent a considerable savings in overall production costs as well as a process that is much more sustainable. In addition, essentially all the carbon in the biogas is fixed as organic carbon products with the instant invention.

By combining the biogas (directly or after reforming via a non-catalytic partial oxidation process) with a syngas from reforming natural gas (or other H₂ rich gas that has a high e⁻/C ratio) there exists the opportunity to fix the CO₂ in the biogas as a Liquid Product. This is done by combining the gases in a ratio to achieve a combined gas e⁻/C of between the optimal range of 6.4 to 5.8.

The remaining digestate from the anaerobic digestion of the high moisture renewable material contains a substantial amount of carbon as solids. Several methods are available for recovery of carbon from this digestate. The preferred method of this invention recovers this carbon through gasification of the solids to produce additional syngas. This option becomes economically viable if there is sufficient waste heat available from the syngas fermentation process to provide much of the drying energy requirements. In this manner the process of the invention will recover essentially of the available carbon from the high moisture organic feedstocks. In addition as described above, employment of this process step will provide additional nutrients as well as the recovered carbon.

In one embodiment of this invention provides processes that can use a single, commercial-scale fermentation vessel to achieve high bioconversion of gas substrate comprising carbon monoxide and hydrogen to Liquid Products usually in the form of oxygenated organic compound by anaerobic fermentation in an aqueous menstruum without undue energy costs and greater economies of processing and feedstock supply. Especially useful economies are achievable through an aspect of this invention that uses a CSTR. Commercial viability is further enhanced as preferred processes of this invention can provide high conversions to oxygenated organic compound employing vessels rated for use essentially at atmospheric pressure. The processes of this invention use a deep fermentation vessel, preferably in the form of a stirred tank reactor having a height of at least about 10, often between about 10 or 15 and 30, meters and an aspect ratio of height to diameter of at least about 0.5:1, preferably between about 0.75:1 to 3:1, with a relatively stable gas-in-water dispersion as the aqueous menstruum generated by injection of the gas feed with a motive liquid. The processes of this invention can further comprise recycling a portion of the off-gas from the aqueous menstruum back to the aqueous menstruum in admixture with fresh gas feed in an amount sufficient to (i) achieve a conversion of the total of carbon monoxide and hydrogen in the gas feed to oxygenated organic compound of at least about 80, preferably at least about 85, often between about 85 and 95, mole percent and (ii) attenuate the risk of carbon monoxide inhibition.

In another aspect this invention can also use high moisture, renewable feedstocks in integrated anaerobic digestion treatment (AD) and synthesis gas (Syngas) fermentation to alcohols and other soluble products. More specifically, the invention can be used to generate a process fuel and a fermentation substrate at increased product yield and reduced cost by use of high moisture, renewable feedstocks such as the organic fraction of municipal solid waste (OFMSW); biological waste sludge; source separated organics; green wastes such as food wastes (FW) from supermarkets, cafeterias, etc.; and other such plentiful, renewable sources such as certain agricultural wastes and even specifically harvested “energy” crops. Thus, the present invention can integrate the anaerobic digestion (AD) of high moisture organic feedstocks such as excess biosolids from a syngas fermenter operation, and additional supplemental outside sources of readily renewable organics substrates to produce a biogas stream. This biogas is then reformed to syngas and blended with the production of a syngas from reforming natural gas (NG) at a resulting mixture that has the e⁻/C ratio necessary to allow H₂/CO₂ and CO to soluble products in the syngas fermentation process.

The AD process can also produce additional fermentation products and water that can be used in the syngas to chemicals fermentation process to potentially supply not only syngas, but also nitrogen (N), phosphorus (P) and H₂S, major nutrients required in the fermentation. Also in one embodiment recovery and reuse of valuable trace metals can be achieved.

Thus, the present invention includes processes the include equipment and arrangements for managing high moisture organic wastes and feedstocks as described herein, that simplifies the treatment and facilitates for their utilization to provide valuable sources of nutrients such as nitrogen, sulfur and phosphorus, as well as CH₄ and CO₂, which are all useful for fermentation of Syngas to soluble oxygenated products.

Another aspect of the invention can include reforming of the biogas can be reformed to syngas using a non-catalytic partial oxidation processing steps and blending the produced syngas with syngas from another source to obtain the desired syngas composition to maximize utilization of all the carbon (CH₄, CO and CO₂) in the combined syngas feed to the Liquid Products generated in the syngas fermentation processes of this invention. The partial reforming technology can include but is not limited to an oxygen fed system of patent application or a plasma arc type gasification process. At least some of the H₂S produced in the AD is conserved in the syngas produced from the biogas and can be used as sulfur source in the syngas fermentation.

In another aspect of the invention, nitrogen and phosphorus are converted to simple inorganic forms (NH₄ and PO₄) in the AD process and are recovered in a form that can be readily used back in the syngas fermentation.

The invention includes another possible aspect wherein the processes can dewater, dry, and gasify the digestate to produce an ash that contains essentially all of the minerals and trace metals required for the syngas fermentation. This ash can be processed to recover the metals in a form that can be reused in the syngas fermentation process making the overall processes more sustainable. The digestate from an Anaerobic Digester (AD), that receives the high moisture organic feedstocks, can potentially provide a source of water for use within or outside of the method operations.

Some of these waste streams and renewable feedstocks may require some form of pretreatment before they can be anaerobically digested (such as removal of contaminating materials, size reduction, thermal pretreatment, etc.,) but many of these wastes and organic streams can be directly treated anaerobically in methanogenic systems to produce a biogas containing primarily CH₄ and CO₂ along with some H₂S. The digestion process also converts the nitrogen and phosphorus in the high moisture organic feedstocks, that are often present in forms that not readily bioavailable to the microorganisms of the fermentation process (such as proteins and organic phosphorus containing compounds), into inorganic forms, such NH₄⁺/NH₃ and ortho PO₄ that are recoverable and bioavailable in the Syngas fermentation process. To prevent/reduce the potential for introducing biological contaminants, heat treatment (pasteurization or sterilization) or physical disruption methods can be employed as pretreatments or post-treatments for the recovered components being reused back in the fermentation.

By this invention processes can include other aspects that provide for the bioconversion of syngas to oxygenated organic compound to a Liquid Products. The processes can produce a variety of Liquid Products which in many of the cases will comprise alcohol, such as ethanol, propanol, and butanol where the supply of reduced sulfur nutrient is integrated into the process. The supply of reduced sulfur nutrient is derived from a metabolic process using feeds streams from the bioconversion of syngas to produce a hydrogen sulfide-containing gas that can be directly provided to the fermentation medium used for the bioconversion of syngas.

Thus in another aspect, reduced sulfur nutrient is obtained as a hydrogen sulfide-containing gas from the metabolic degradation of biosolids obtained from the fermentation medium used for the bioconversion of syngas. Often, at least about 30, and sometimes up to 50 percent or more of the sulfur nutrient for the bioconversion of syngas can be recovered from the biosolids recovered from the fermentation medium. This recovery of sulfur nutrient represents a significant savings itself. However, the processes of this invention enable sulfur nutrient to be generated from readily available, less toxic and less expensive sources of sulfur, namely sulfoxy moieties. Where used in combination, substantially the entire reduced sulfur nutrient requirements for the bioconversion of syngas can be achieved. These aspects of the invention may be used singularly or preferably in combination. In the aspect of the invention where sulfoxy moieties are metabolized to hydrogen sulfide, adequate electron donor may inherently be provided by the biomass from which hydrogen sulfide is recovered, and in the absence of, or in addition to, the presence of biomass, at least one of syngas and off-gas provides electron donor.

Through the integration of the reduced sulfur aspects the invention the processes are able to provide reduced sulfur to the fermentation medium for the conversion of syngas in an economically attractive manner through the use of highly efficient bioreactor arrangement. Especially attractive embodiments of the integrated sulfur supply of invention enhance the yield of an oxygenated organic compound and can provide a methane-containing biogas with a low hydrogen sulfide concentration.

Thus in accordance with a further integrated aspects of the processes of this invention, solid debris including microorganisms used for the bioconversion of syngas to oxygenated organic compound are subjected to anaerobic digestion which provides a biogas containing hydrogen sulfide as well as other components such as carbon dioxide and water vapor. The biogas is directly provided to the aqueous fermentation broth for the syngas bioconversion. As the volume of the biogas is relatively small in comparison to the volume of syngas being introduced into the fermentation broth, the operation of the fermentation to bioconvert syngas is not adversely affected. Moreover, as the hydrogen sulfide is dilute in the biogas, reduced risks in handling and introduction into the fermentation broth are obtained. The gases diluting the hydrogen sulfide can pass through the fermentation broth and be ultimately discharged as a tail gas.

A metabolic process, which may or may not be an anaerobic digestion, may be used to convert oxidized forms of sulfur (sulfoxy moieties) or elemental sulfur to hydrogen sulfide. Electron donor for the metabolic process to bioconvert sulfoxy moieties to hydrogen sulfide is derived from the bioconversion of syngas process such as off gas containing at least one of unreacted hydrogen and carbon monoxide or aqueous streams or biosolids derived from the fermentation medium for the bioconversion of syngas. Advantageously syngas or gas effluent from the syngas fermentation is passed to the metabolic process to convert sulfoxy moieties in that not only is electron donor provided but also the gas serves as a sweep gas and dilutes the hydrogen sulfide.

Thus another aspect of the processes of this invention pertains to processes for bioconversion of syngas to oxygenated organic compound with integrated hydrogen sulfide supply and will include the steps of: passing syngas into a syngas reactor containing aqueous fermentation broth under fermentation conditions, the fermentation broth containing microorganisms adapted for bioconverting syngas to oxygenated organic compound, to produce oxygenated organic compound dissolved in the fermentation broth and an off gas; removing from the syngas reactor at least an aliquot portion of the fermentation broth containing oxygenated organic compound and containing biosolids; separating from the aliquot portion of the fermentation broth an aqueous biosolids-containing phase containing biosolids having a higher solids content and a reduced oxygenated organic compound concentration than the aliquot portion; subjecting the biosolids-containing phase to conditions to biodegrade solids in the aqueous liquid phase to provide an aqueous degraded solids product and a biogas product comprising hydrogen sulfide; and, passing at least an aliquot portion, say, at least about 75 volume percent to preferably substantially all, of the biogas to the syngas reactor to provide at least a portion of sulfur nutrient for the microorganisms.

The sulfoxy moiety or elemental sulfur may be supplied in an amount sufficient to provide a biogas containing the sought amount of hydrogen sulfide to meet the nutrient needs of the microorganisms in the reactor. Sulfoxy moieties include, but are not limited to sulfur dioxide, sulfamide and oxyanions of sulfur such as sulfate, sulfite, sulfamate and thiosulfate. Where the sulfoxy moiety is provided by sulfuric acid or sulfurous acid, maintaining the sought pH is facilitated.

The anaerobic digestion conditions to provide a biogas comprising hydrogen sulfide may be methanogenic or acidogenic. Methanogenic digestion is typically operated at a pH of between about 6.8 and 7.6. Acidogenic digestion conditions are frequently preferred for the anaerobic digestion to produce the hydrogen sulfide-containing biogas. Acidogenic digestion conditions generally do not produce methane, but rather provide a degradation to organic acids such as acetic acid. The acidogenic digestion thus provides several advantages. First, the biogas will not be diluted with methane. Although methane is inert in the syngas fermentation and would be a very small component of the tail gas from the reactor, the acidogenic digestion allows biosolids to be treated in a subsequent methanogenic, anaerobic digester to provide a biogas having a higher energy density and lower sulfur content. Second, the organic acids generated in the acidogenic digestion may be recovered and passed to the syngas reactor for bioconversion to oxygenated organic compound. Third, usually acidogenic digestion conditions provide for a greater conversion of sulfur contained in the biomass or as provided by sulfoxy moieties to hydrogen sulfide as opposed to HS⁻ by maintaining the pH more acidic than the pKa of hydrogen sulfide. Typically acidogenic conditions comprise a pH of about 6, say 4.5 or 5 to 6.

Preferably where an acidogenic conditions are used for the anaerobic digestion, the aqueous degraded solids product is subjected to a subsequent anaerobic, methanogenic digestion to provide a biogas containing methane. As hydrogen sulfide has been removed during the acidogenic fermentation, the biogas from the methanogenic digestion can be relative free of hydrogen sulfide and thus may be directly useful as a gas to generate heat by combustion or to power engines. In some instances, the concentration of hydrogen sulfide in the methane-containing biogas is less than about 100, preferably less than about 20, parts per million by volume (ppmv).

Therefore, another broad aspect of the processes of this invention can incorporate into processes for bioconversion of syngas to oxygenated organic compound an integrated hydrogen sulfide supply that comprises: providing a sulfoxy moiety to a sulfoxy bioreactor containing an aqueous metabolizing broth containing microorganisms capable of reducing sulfoxy moiety to hydrogen sulfide in the presence of electron donor, the metabolizing broth being at metabolizing conditions and providing a hydrogen sulfide-containing biogas; providing syngas to a syngas bioreactor containing an aqueous fermentation medium capable of bioconverting syngas to oxygenated organic compound, the fermentation medium being at fermentation conditions to provide a fermentation broth containing oxygenated organic compound and biosolids and to provide an off gas containing at least one of hydrogen and carbon monoxide; providing to the sulfoxy bioreactor an electron donor from the fermentation bioreactor, preferably at least one of an aliquot portion of the syngas to be provided to the syngas bioreactor, at least an aliquot portion of the off gas from the syngas bioreactor, at least a portion of the biosolids contained in the syngas bioreactor, and at least a portion of the fermentation medium from the syngas bioreactor, in an amount sufficient to provide electron donor to provide the hydrogen sulfide containing gas; and, passing at least an aliquot portion of the hydrogen sulfide-containing biogas to the syngas bioreactor.

In another important aspect of the invention, by recycling a portion of the off-gas for admixture with fresh gas feed being passed to the reactor, the composition of the gas bubbles can be adjusted such that the rate of mass transfer to the aqueous menstruum does not unduly exceed the rate of bioconversion of carbon monoxide and thereby avoid carbon monoxide inhibition. In suitable form the processes of this invention achieve high conversion efficiencies of carbon monoxide and hydrogen, the off-gases at steady state operating conditions will have a low mole fraction of carbon monoxide and hydrogen and thus be effective for controlling the composition of the gas bubbles being passed to the reactor.

The sulfur supply aspect of this invention includes the possibility of adding to the processes a reliable, cost-effectively and efficiently supply of sulfur nutrient to microorganisms contained in acidic, aqueous fermentation menstrua. In preferred embodiments, the supply of sulfur nutrient occurs without undue concentrations of hydrogen sulfide being contained in off-gases from the aqueous menstruum. In accordance with one aspect of the processes of this invention, at least a portion of the sulfur nutrient for the population of microorganisms in the aqueous menstruum is supplied as calcium sulfite, and the presence of undissolved calcium sulfite is maintained in the aqueous menstruum and, in essence, serves as a reservoir for sulfur nutrient supply.

The presence of undissolved calcium sulfite assures a continuing supply of a low concentration of sulfite anion to the aqueous menstruum. Without wishing to be limited to theory, it is believed that the solubility equilibria of calcium sulfite is such that so long as undissolved calcium sulfite remains in the aqueous menstruum, a dissolved calcium sulfite concentration of between about 20 and 50 milligrams per liter can be maintained in the aqueous menstruum. At these low concentrations, the microorganisms are still able to obtain the needed sulfur nutrient while not generating undue amounts of hydrogen sulfide.

In this aspect of adding calcium sulfite the continuous processes of this invention for converting the substrate to oxygenated organic compound, the processes comprise: continuously contacting the gas substrate with the aqueous menstruum under acidic, anaerobic fermentation conditions to bioconvert gas substrate to oxygenated organic compound and provide an oxygenated organic compound-containing menstruum and a depleted gas phase; continuously withdrawing the depleted gas phase from the aqueous menstruum; continuously or intermittently withdrawing a portion of the menstruum for recovery of the oxygenated organic compound, the withdrawal being sufficient to maintain the oxygenated organic compound in the menstruum below a concentration that unduly adversely affects the microorganisms, wherein during the contacting the aqueous menstruum contains undissolved calcium sulfite.

In the aspect of adding calcium sulfite the processes include continuously contacting the gas substrate with the aqueous menstruum under acidic anaerobic fermentation conditions to bioconvert gas substrate to oxygenated organic compound and provide an oxygenated organic compound-containing menstruum and a depleted gas phase and continuously withdrawing the depleted gas phase from the aqueous menstruum. The processes continuously or intermittently withdraw a portion of the menstruum for recovery of the oxygenated organic compound, the withdrawal being sufficient to maintain the oxygenated organic compound in the menstruum below a concentration that unduly adversely affects the microorganisms, wherein during the contacting undissolved calcium sulfite is provided to the aqueous menstruum in an amount sufficient to maintain undissolved calcium sulfite in the aqueous menstruum. Preferably, the undissolved calcium sulfite is suspended in the aqueous menstruum and is in substantial uniformity in the liquid phase. Despite any acidity, the depleted gas phase often contains less than about 100, and most frequently less than about 50, parts per million by volume (ppmv) of hydrogen sulfide where calcium sulfite provides substantially all of the sulfur nutrient to the aqueous menstruum.

In other aspects of the invention the calcium sulfite may provide all or a portion of the sulfur requirements for the microorganisms in the processes. In many aspects, the calcium sulfite, other than indigenous sulfur compounds inherently contained in the feed gas supplying the gas substrate, provides at least about 50 percent, more preferably at least about 80 percent to substantially all, of the sulfur nutrient requirements. The calcium sulfite can also be used as a reservoir of sulfur nutrient source in the event that a disruption in the supply of other sulfur nutrient to the aqueous menstruum occurs. The calcium sulfite can be provided to the aqueous menstruum as calcium sulfite, e.g., as solids or as solids slurried in a suitable liquid, preferably an aqueous liquid, which is not deleterious to the microorganisms. Alternatively or in addition, a soluble salt of sulfite, e.g., an alkali metal salt, including but not limited to, sodium and potassium, and ammonium salt, can be used and precipitated with calcium cation in the aqueous menstruum (in situ precipitate). In other preferred aspects of the processes of this invention at least 70, and most preferably essentially all, the calcium sulfite is added to the aqueous menstruum in the form of an aqueous slurry.

Usually in the applicable aspects calcium sulfite solids in the aqueous menstruum are in the form of finely divided particles, e.g., having a major dimension of up to about 100 microns. These small particulates provide a high surface area per unit of mass to facilitate maintaining the equilibrium between undissolved calcium sulfite and dissolved calcium sulfite. In some instances, the undissolved calcium sulfite may be colloidal particles. Preferably essentially all the undissolved calcium sulfite is of sufficiently small particle size that it can be maintained in a relatively uniform dispersion in the aqueous menstruum. Consequently, where solid calcium sulfite is added to the aqueous menstruum, preferably at least about 50 mass percent of the solids have a maximum particle size dimension of between about 1 and 100 microns.

The calcium sulfite or precursors for the precipitation of calcium sulfite can be added continuously or intermittently to the aqueous menstruum. So long as undissolved calcium sulfite is present, as the concentration of dissolved calcium sulfite anion below the saturation concentration at the conditions of the aqueous menstruum, a driving force exists to cause solubilization of more calcium sulfite. Accordingly, the concentration of undissolved calcium sulfite in the aqueous menstruum is not critical and can vary over a wide range. One advantage of the processes of this invention is that the concentration of undissolved calcium sulfite need not be monitored to match the rate of metabolism of the sulfite anion. Often, the concentration of undissolved calcium sulfite is between about 50 milligrams per liter to about 1 gram or more per liter.

In a stirred tank aspect of this invention the fermentation reactor uses one or more mechanical stirrers and provides a beneficial ratio of energy for mechanical stifling to volume. Preferably the mechanical stifling is at a rate insufficient to cause undue agglomeration of gas phase microbubbles. The mechanical stirring should be sufficient to promote the uniformity of liquid composition through the reactor and need not, and preferably is not, used as a generator of a significant fraction of the microbubbles. For purposes herein the type of stirred tank reactor used in the processes of this invention is called a mechanically-assisted liquid distribution (MLD) tank reactor. With the relatively uniform composition throughout the MLD tank reactor provided by the mechanical stirring, regardless of where the gas feed is introduced, bubbles will be moved throughout the volume of the aqueous menstruum. Where the invention uses a motive fluid for instance in a venturi or jet injector, to generate the microbubbles for the dispersion, rather than the mechanical stifling, energy savings are realized. Moreover, the injectors can provide better control over the size of the gas bubbles being introduced into the aqueous menstruum and thus the interfacial area between the gas and liquid phases. Changing bubble size thus modulates the mass transfer of carbon monoxide and hydrogen to the aqueous menstruum. Since the mechanical stifling does not adversely affect the gas bubbles, the modulation achieved by adjusting bubble size provides a viable control of the process.

Therefore, in another aspect, the processes of this invention can comprise: the anaerobic bioconversion of a gas substrate comprising carbon monoxide and hydrogen in an aqueous menstruum kept under anaerobic conditions and containing microorganisms suitable for bioconverting the substrate to oxygenated organic compound in a deep, continuously-stirred tank bioreactor that maintains continuous mechanical stifling of an aqueous menstruum containing the microorganisms, the aqueous menstruum having an upper portion with a head space above the upper portion and a lower portion and having a depth of at least 10 meters in the reactor; continuously supplying a gas substrate as a gas feed to the aqueous menstruum by injection using a motive liquid to form a stable gas-in-liquid dispersion in the aqueous menstruum; bioconverting carbon monoxide and hydrogen and carbon dioxide to Liquid Products, oxygenated organic compounds, providing off-gas from the aqueous menstruum in the head space, and withdrawing from the head space at least a portion of the off-gas; and admixing at least a portion of the withdrawn off-gas with the gas substrate in an amount sufficient to (i) achieve a bioconversion efficiency of the total of carbon monoxide and hydrogen in the gas substrate to oxygenated organic compound of at least about 80 mole percent and (ii) attenuate the risk of carbon monoxide inhibition of the microorganism used for the bioconversion. In this aspect the rate of mechanical stifling provides relatively uniform liquid phase composition within the aqueous menstruum without unduly adversely affecting the gas-in-liquid dispersion. Often, in this form of the inventions the energy required for the mechanical stifling is less than 0.02 watt per liter of aqueous menstruum.

The stable gas-in-water dispersion is provided using microbubbles of gas, preferably less than 500, more preferably less than 300, say about 10 or 20 to 300, microns in diameter. Suitable devices for generating and introducing the gas-in-liquid dispersions include venturi injectors, jet injectors and, preferably, slot injectors, where the motive liquid contains oxygenated organic compound or other surface active agent. As the size of the microbubbles can be varied by changing the rate of flow of the motive liquid, an additional means for control of the mass transfer of gas substrate to the liquid phase can be achieved. Jet injectors, and especially slot injectors, can provide a suitable sized bubble to enable the stable dispersion to be formed while maintaining a surface area to volume ratio to provide a rate of transfer high enough to obtain desired efficiencies of conversion but low enough to avoid carbon monoxide inhibition.

In a preferred aspect of the invention, the rate of supply of fresh gas feed for admixing with recycled off-gas is controlled in response to the conversion efficiency. In this aspect, the rate that carbon monoxide and hydrogen transfer to the liquid phase can readily be adjusted to reflect the conditions of the aqueous menstruum, thereby optimizing conversion of gas substrate while avoiding the risk of carbon monoxide inhibition. The rate of transfer of carbon monoxide to the liquid phase would therefore be in concert with the rate that the colony of microorganisms can bioconvert the carbon monoxide, i.e., no build-up of carbon monoxide concentration would occur in the aqueous menstruum.

The motive liquid may be any suitable aqueous liquid for introduction into the aqueous menstruum including make-up water, aqueous streams from product recovery, aqueous streams recovered from the purge of solids and recycled aqueous menstruum. In a preferred aspect of the invention, at least a portion of the motive, aqueous liquid is derived from aqueous menstruum withdrawn from the reactor. In one embodiment, the introduction of gas feed is accomplished at a lower portion of the reactor and aqueous menstruum for recycle is withdrawn from an upper portion of the reactor. Although the composition of the aqueous menstruum is relatively uniform throughout the reactor, this embodiment takes advantage of relatively small compositional differences. In other embodiments, the gas feed is supplied at two or more heights in the reactor.

### BRIEF DESCRIPTION OF THE DRAWINGS

FIG. 1 provides a generic, schematic diagram of one embodiment of the proposed process aspects associated with the use of the methodology presented herein below.

FIG. 2 provides a generic, schematic diagram of a further embodiment of the proposed process aspects associated with the use of the methodology presented herein below

FIG. 3 provides a generic, schematic diagram with a yet different embodiment of the proposed process aspects associated with the use of the methodology presented herein below.

FIG. 4 is a schematic flow diagram of a deep, MLD tank reactor adapted to use the process of this invention.

FIG. 5 is a schematic depiction of the process aspects for obtaining sulfur supply in the process of this invention.

FIG. 6 is a schematic depiction of a modification to the process aspects of FIG. 5.

FIG. 7 is a schematic depiction of an apparatus that can be used in the aspect of this invention that provides removal of an Adverse Component.

### DETAILED DESCRIPTION OF THE INVENTION

All patents, published patent applications, unpublished patent applications and articles referenced herein are hereby incorporated by reference in their entirety.

### DEFINITIONS

As used herein, the use of the terms “a” and “an” is intended to include one or more of the element described.

As used herein, the term oxygenated organic compound means one or more organic compounds containing two to six carbon atoms selected from the group of aliphatic carboxylic acids and salts, alkanols and alkoxide salts, and aldehydes. Often oxygenated organic compound is a mixture of organic compounds produced by the microorganisms contained in the aqueous menstruum. Preferred oxygenated organic compounds are ethanol, n-propanol, i-propanol, n-butanol, i-butanol and acetone.

As used herein the term nitrate anion includes an anion derived from one or more water-soluble nitrate salts, nitric acid and mixtures thereof.

As used herein the Adverse Component comprises one or more oxygenated organic compounds other than the product oxygenated organic compound. The Adverse Component, i.e., co-produced oxygenated organic compound, is one or more metabolic products from the bioconversion of syngas which may be produced by the microorganism producing the product oxygenated organic compound or an adventitious microorganism.

As used herein a bioreactor assembly is an assembly of one or more vessels suitable to contain aqueous broth and microorganisms for the bioconversion and can contain associated equipment such as injectors, recycle loops, agitators, and the like.

As used herein chemical oxygen demand (COD) is the amount of oxygen required to convert organic carbon in water to carbon dioxide and thus is an indication of the organic compound content of the water. COD is reported as milligrams per liter. One procedure for determining COD is Hach Method 8000, February 2009, Ninth Edition.

As used herein biomass means biological material living or recently living plants and animals and contains at least hydrogen, oxygen and carbon. Biomass typically also contains nitrogen, phosphorus, sulfur, sodium and potassium. The chemical composition of biomass can vary from source to source and even within a source. Sources of biomass include, but are not limited to, harvested plants such as wood, grass clippings and yard waste, switchgrass, corn (including corn stover), hemp, sorghum, sugarcane (including bagas), and the like; and waste such as garbage and municipal waste. Biomass does not include fossil fuels such as coal, natural gas, and petroleum.

The abbreviation ppm means parts per million. Unless otherwise stated or clear from the context, ppm is on a mass basis (ppm (mass)) for solids in a liquid medium.

As used herein fossil carbonaceous materials, or fossil fuels, include, but are not limited to, natural gas; petroleum including carbonaceous streams from the refining or other processing of petroleum including, but not limited to, petroleum coke; and lignite and coal.

As used herein the term syngas is a gas containing carbon monoxide and frequently hydrogen, although term “syngas”, for purposes herein, is also intended to encompass carbon monoxide gas streams that may have little or no hydrogen. Typically, carbon monoxide is present in an amount of at least about 20 volume percent, and the syngas typically contains other components in addition to hydrogen such as carbon dioxide, nitrogen and water vapor. Syngas may derived from various sources, including, but not limited to, gasification of carbonaceous feedstocks such as biomass, landfill gas, coal, natural gas, and petroleum; coke oven gas and gas from other industrial operations such as petroleum refining and steel mill waste gas.

As used herein uniformity in the liquid phase means that the composition of the aqueous menstruum is relatively uniform throughout the deep, MLD reactor. Uniformity can be determined by measuring the concentration of oxygenated organic compound in samples taken at a lower portion and at an upper portion of the aqueous menstruum, and uniformity exists if the concentration of the oxygenated organic compound in the samples does not vary by more than 20 mole percent.

As used herein the motive liquid may be any suitable liquid for introduction into the reactor. The motive liquid comprises sufficient amount of one or more of oxygenated organic compound and other surface active agent to enhance the formation of microbubbles.

As used herein microbubbles are bubbles having a diameter of 500 microns or less.

As used herein the pressure at the point of injection into the aqueous menstruum is the sum of the absolute pressure at the point calculated as if the liquid head above such point were water. The partial pressure of a gas feed component is determined as the product of the mole fraction of a component in a gas mixture times the total pressure. The partial pressure of a component in the gas being fed to a reaction reactor is calculated as the mole fraction of that component times the pressure in the reaction reactor at the point of entry.

As used herein stable gas-in-liquid dispersion means a mixture of gas bubbles in liquid where (i) the bubbles predominantly flow in the same direction as the liquid, and (ii) the dispersion is sufficiently stable that it exists throughout the aqueous menstruum, i.e., insufficient coalescing of bubbles occurs to destroy the dispersion.

As used herein carbon monoxide inhibition means that microorganisms are adversely affected by a high concentration of dissolved carbon monoxide in the aqueous menstruum resulting in a significantly reduced, e.g., reduced by at least 5 percent, conversion of carbon monoxide or hydrogen per gram of active cells per liter, all other conditions remaining the same. The inhibitory effect may occur in a localized region in the aqueous menstruum; however, the occurrence of a carbon monoxide inhibition is typically observed by assessing the overall conversion for the volume of aqueous menstruum in the reactor. The concentration of carbon monoxide dissolved in the aqueous menstruum that results in carbon monoxide inhibition varies depending upon the strain of microorganism and the fermentation conditions.

As used herein Liquid Products means alcohol and acids such as acetic acid, propionic acid and butyric acid.

As used herein Electron to carbon ratio is calculated as the quotient of the quantity of two times the sum of the molar concentrations of carbon monoxide and hydrogen divided by quantity of the sum of the molar concentrations of carbon monoxide and carbon dioxide:

e⁻/C=2([CO]+[H2])/([CO]+[CO2]).

As used herein fermentation broth aqueous broth, or aqueous fermentation broth, means a liquid water phase which may contain dissolved compounds including, but not limited to hydrogen, carbon monoxide, and carbon dioxide. The broth may, but is not required, to contain microorganisms and may contain required nutrients including N, P, and S, dissolved salts, and trace metals required for the optimal growth of the syngas fermenting organisms.

As used herein intermittently means from time to time and may be at regular or irregular time intervals.

As used herein syngas means a gas, regardless of source, containing at least one of hydrogen and carbon monoxide and may, and usually does, contain carbon dioxide.

As used herein Alcohol means one or more alkanols containing two to six carbon atoms. In some instances alcohol is a mixture of alkanols produced by the microorganisms contained in the fermentation broth. The most common alcohols produced by this method are ethanol and butanol.

As used herein a concentration of Liquid Products, (primarily alcohol) below that which unduly adversely affects the rate of growth of the culture of microorganisms will depend upon the type of microorganism and the alcohol. An unduly adverse effect on the growth rate means that a significant, usually at least a 20 percent, decrease in the growth rate of the microorganisms is observed in comparison to the growth rate observed in an fermentation broth having about 10 grams per liter alcohol therein, all other parameters being substantially the same.

As used herein substantial uniformity of a component in liquid phase means that the concentration of that component in the liquid phase is substantially the same throughout a bioreactor. Usually the concentration of the component is within about 0.2 mole percentage points in a uniform liquid phase.

As used herein deep tank bioreactor is a bioreactor having a depth of at least about 10 meters and can be operated to provide a substantial non-uniform substrate composition over the depth of the aqueous menstruum contained in the bioreactor.

As used herein the term bubble column bioreactor refers to a deep tank bubble column bioreactor unless otherwise explicitly stated and include deep tank reactors where the gas is introduced as small bubbles to promote mixing.

As used herein a commercial scale bioreactor has a capacity for aqueous menstruum of at least 1 million, and more preferably at least about 5, say, about 5 to 25 million, liters.

As used herein substrate is one or more of (i) carbon monoxide and (ii) carbon dioxide and hydrogen. A feed gas contains substrate and may contain other components including, but not limited to, recycled off-gas or a fraction thereof and other additives, inerts such as methane and nitrogen.

Overview

The processes of this invention pertain to operating deep fermentation reactors, such as BCBRs, stirred tank fermentation reactors, particularly deep, MLD tank reactors, for anaerobic bioconversion conversion of a gas substrate containing carbon monoxide, hydrogen and carbon dioxide to produce an oxygenated organic compound in the form of Liquid Products and may in various aspects include: removing an Adverse Component from an aqueous fermentation broth by adding nitrate anion to the fermentation broth; using denitrifying microorganisms to degrade the Adverse Component in the denitrification; and providing reliable, cost-effective and efficient supply of sulfur nutrient to the deep tank fermentation reactors.

Substrate and Feed Gas

Anaerobic fermentation to produce oxygenated organic compound uses a substrate (syngas) comprising at least one of (i) carbon monoxide and (ii) carbon dioxide and hydrogen, the latter being for the hydrogen conversion pathway. The feed gas will typically contain nitrogen and methane in addition to carbon monoxide and hydrogen. Syngas is one source of a gas substrate. Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas.

Syngas is typically produced by a gasifier or reformer (steam, autothermal or partial oxidation). Any of the aforementioned biomass sources are suitable for producing syngas. The syngas produced thereby will typically contain from 10 to 60 mole % CO, from 10 to 25 mole % CO₂ and from 10 to 60 and sometimes 75 mole % H₂, often at least about 30, and preferably between about 35 and 65, mole % H₂. The syngas may also contain N₂ and CH₄ as well as trace components such as H₂S and COS, NH₃ and HCN. Other compositions of syngas contain 25 to 70, say, 40 to 65, mole percent carbon monoxide; 0 to 70, say, 30 or 40 to 65, mole percent hydrogen; and 1 to 20, say 3 to 15, mole percent carbon dioxide excluding nitrogen and water vapor from the concentration calculations. Other sources of the gas substrate include gases generated during yeast fermentations; petroleum and petrochemical processing; and industrial processes. These gases may have substantially different compositions than typical syngas, and may be essentially pure hydrogen or essentially pure carbon monoxide. The gas substrate may be obtained directly from gasification or from other sources including, but not limited to those previously disclosed or by blending two or more such streams. Also, the gas substrate may be treated to remove or alter the composition including, but not limited to, removing components by chemical or physical sorption, membrane separation, and selective reaction. In addition other components may be added to the gas substrate such as nitrogen or adjuvant gases such as ammonia and, as described herein, hydrogen sulfide.

The source of the syngas is not critical to the broad aspects of this invention. The syngas should, however, be free of components in concentrations that would be unduly adverse to the microorganisms used in the fermentation such as, but not limited to, hydrogen cyanide, alkenes, and alkynes and that would be adverse if present in the sought oxygenated organic compound such as tars and aromatics where ethanol is the sought product.

For the sake of ease of reading, the term syngas will be used herein and will be intended to include these other gas substrates.

In accordance with the processes of this invention, a portion of the off-gas from above the top of the aqueous menstruum may be admixed with fresh gas feed, or syngas, to enable high conversion of gas substrate to oxygenated organic compounds and to attenuate the risk of carbon monoxide inhibition of the microorganisms. The composition of the mixture will have a lower mole fraction of carbon monoxide and hydrogen than that in the syngas due to the presence of carbon dioxide contained in the recycle gas as well as inert or other gases that may be contained in the syngas such as nitrogen and methane. Since a portion of the off-gas is recycled, inert and other gases will build up to a steady-state composition.

The off-gases will contain some unreacted carbon monoxide and hydrogen. The portion of the off-gases that will be recycled to the aqueous menstruum will be sufficient to provide the required molar conversion efficiency of this invention. Accordingly, capital and operating costs associated with an additional reactor in series need not be incurred to provide commercially-attractive conversion efficiencies. The operator can vary one or both of the recycle rate of off-gases and the feed rate of fresh syngas to achieve a desired conversion efficiency.

The common commercial expectation is that the fermentation process will be operated to obtain a production rate of oxygenated organic compound that provides the greatest margin, i.e., the lowest fixed and variable cost per unit of production. As the cost of syngas is expected to be the primary cost driver, the operator has flexibility to operate the process to maximize margin as market conditions then exist. Aggressive production regimes can be used as the risk of carbon monoxide inhibition is attenuated by the recycle of off-gases. The operator can thus match the bioconversion capacity of the culture of microorganisms in the aqueous menstruum with the rate of transfer of carbon monoxide and hydrogen to the aqueous menstruum subject to equipment and energy limitations.

Due to this flexibility, the volume ratios of fresh syngas to recycled off-gases can vary widely. And these ratios will change should an event occur that adversely affects to productivity of the culture of microorganisms in the aqueous menstruum. The ratios are generally in the range of about 0.5:10 to 10:1, preferably, 1:5 to 5:1, cubic meter of recycled off-gas per cubic meter of fresh syngas at standard temperature and pressure. Frequently the gas feed compositions to the injectors are as set forth in the following table:

| Component | Usual, mole percent | Preferred, mole percent |
|---|---|---|
| Carbon monoxide | 5 to 50 | 10 to 35 |
| Hydrogen | 5 to 50 | 10 to 35 |
| Carbon dioxide | 10 to 70 | 10 to 50 |
| Nitrogen | 0 to 20 | 0 to 10 |
| Methane | 0 to 10 | 0 to 5 |

The portion of the off-gas not recycled, can be sent to recovery of any contained oxygenated organic compound and the remaining energy content recovered, e.g., by combustion in, for instance, a device such as a thermal oxidizer. The ratio of recycled to exhausted off-gas can vary widely depending upon the sought conversion of syngas to oxygenated organic compound. Practical limits exist to the conversion efficiencies that can be achieved in commercial operations. For instance, the exhaust stream should be sufficient to maintain inerts and other components in the off-gas and in the gas feed at acceptable levels.

The substrate depleted gas phase egressing from the aqueous fermentation broth will contain a small fraction of the hydrogen and carbon oxides introduced into the bioreactor assembly as the feed gas. Inerts such as nitrogen and primarily methane will comprise a portion of the depleted gas phase where syngas from steam reforming or oxygen-fed, autothermal reforming, especially steam or autothermal reforming of methane-containing gas, is used. The depleted gas phase may also contain sulfur-containing compounds, alcohol and the like volatilized from the aqueous fermentation broth.

The recycled off-gases may be treated to remove a portion of the carbon dioxide prior to admixture with fresh syngas. Any suitable carbon dioxide removal process may be used including amine extraction, alkaline salt extractions, water absorption, membrane separation, adsorptions/desorption, and physical absorption in organic solvents. A preferred process for removal of carbon dioxide from gases is by contacting the gas with an aqueous solution containing oxygenated organic compound. This process for removing carbon dioxide from gas to be fed to a fermentation zone, including between sequential fermentation stages, is disclosed in U.S. Patent application No. 2008/0305539, filed Jul. 23, 2007. See also, U.S. patent application Ser. No. 12/826,991, filed Jun. 30, 2010, which discloses contacting a gas stream with a mixture of water and a surface active agent under pressure to sorb carbon dioxide and phase separating the gas and liquid stream to provide a gas stream with reduced carbon dioxide concentration to be used a feed to a fermentation zone. US 2008/0305539 A1 discloses the use of membranes to remove carbon dioxide from a membrane supported fermentation system to prevent dilution of concentrations of carbon monoxide and hydrogen in a multistage system.

If desired, a portion of the carbon dioxide dissolved in the liquid phase of the aqueous menstruum can be removed. Any convenient unit operation for carbon dioxide removal can be used, but the preferred operation is separation by reducing the pressure to atmospheric or lower pressure to flash carbon dioxide gas from the liquid phase.

Oxygenated Compounds and Microorganisms

The oxygenated organic compounds produced in the processes of this invention will depend upon the microorganism or combination of microorganisms used for the fermentation and the conditions of the fermentation reactor. One or more microorganisms may be used in the fermentation menstruum to produce the sought oxygenated organic compound. Bioconversions of CO and H₂/CO₂ to acetic acid, n-propanol, butanol, butyric acid, ethanol and other products are well known. For example, a description of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds., Springer (2003). Any suitable microorganisms that have the ability to convert the syngas components: CO, H₂, CO₂ individually or in combination with each other or with other components that are typically present in syngas may be utilized. Suitable microorganisms and/or growth conditions may include those disclosed in U.S. patent application Ser. No. 11/441,392, filed May 25, 2006, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 described in U.S. Pat. No. 8,143,037.

Mixed cultures of anaerobic microorganisms can also be used for the bioconversions of syngas to product oxygenated organic compounds. See, for instance, U.S. patent application Ser. No. 13/802,916, filed Mar. 14, 2013, entitled Method for production of n-propanol and other C3-carbon containing products from syngas by symbiotic arrangement of C1-fixing and C3-producing anaerobic microorganism cultures (Toby, et al.); Ser. No. 13/802,930, filed Mar. 14, 2013, entitled method for production of n-propanol and/or ethanol by fermentation of multiple substrates in a symbiotic manner (Enzein, et al. C1-fixing microorganisms include, without limitation, homoacetogens such as Clostridium ljungdahlii, Clostridium autoethanogenum, Clostridium ragsdalei, and Clostridium coskatii. Additional C1-fixing microorganisms include Alkalibaculum bacchi, Clostridium thermoaceticum, and Clostridium aceticum. Symbiotic C3-producing microorganisms capable of growing on ethanol and/or acetate as their primary carbon source include, but are not limited to, Pelobacter propionicus, Clostridium neopropionicum, Clostridium propionicum, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosymbioticum, Pelotomaculum thermopropionicum, and Pelotomaculum schinkii. Pathways for the production of product oxygenated organic compounds having three carbons include, but are not limited to, Propionibacterium species (Propionibacterium acidipropionici, Propionibacterium acnes, Propionibacterium cyclohexanicum, Propionibacterium freudenreichii, Propionibacterium freudenreichii shermanii, Propionibacterium pentosaecum) and several other anaerobic bacteria such as Desulfobulbus propionicus, Pectinatus frisingensis, Pelobacter propionicus, Veillonella, Selenomonas, Fusobacterium, Bacteroides fragile, Prevotella ruminicola, Megasphaera elsdenii, Bacteroides vulgates, and Clostridium, in particular Clostridium propionicum.

Aqueous Menstruum and Fermentation Conditions:

The aqueous menstruum will comprise an aqueous suspension of microorganisms and various media supplements. Suitable microorganisms generally live and grow under anaerobic conditions, meaning that dissolved oxygen is essentially absent from the fermentation liquid. The bioreactor may have added from time to time or continuously one or more streams of water, nutrients or adjuvants, and microorganisms. The various adjuvants to the aqueous fermentation broth may comprise buffering agents, trace metals, vitamins, salts etc. Adjustments in the fermentation broth may induce different conditions at different times such as growth and non-growth conditions which will affect the productivity of the microorganisms. U.S. Pat. No. 7,704,723 discloses the conditions and contents of suitable aqueous fermentation broth for bioconversion CO and H₂/CO₂ using anaerobic microorganisms.

The top of the deep, MLD tank reactor may be under pressure, at atmospheric pressure, or below ambient pressure. Preferably the fermentation is conducted at substantially atmospheric pressure, for instance with a pressure at the top of less than about 30 kPa gauge, to reduce capital cost of the reactor. The menstruum is maintained under anaerobic fermentation conditions including a suitable temperature, say, between 25 C and 60 C, frequently in the range of about 30 C. to 40 C. The conditions of fermentation, including the density of microorganisms, aqueous menstruum composition, and aqueous menstruum compostion and depth are preferably sufficient to achieve the sought conversion efficiency of hydrogen and carbon monoxide. The pH of the aqueous menstruum or broth, undergoing treatment or otherwise, is often less than about 6.5, say, between about 4 and 6.0, and most frequently between about 4.5 and 5.5 as lower pH environments favor solventogenenesis. Although the aqueous fermentation broth can be at higher pH levels, e.g., 8 or 9 or more, preferably the pH is maintained below about 7.0 or 7.5 to minimize the adjustment required to reestablish the desired, acidic pH for the syngas bioconversion.

The rate of supply of the feed gas under steady state conditions to a fermentation broth or each of the primary and/or sequential reactors is preferably such that the rate of transfer of carbon monoxide and hydrogen to the liquid phase matches the rate that carbon monoxide and hydrogen are bioconverted. Hence, in this aspect of operation the dissolved concentration of carbon monoxide and hydrogen in the aqueous phase remains constant, i.e., does not build-up.

The average residence time of the gas in the fermentation zone will depend upon the depth of the aqueous menstruum and the size of the microbubbles and the internal fluid flows in the vessel cause by the mechanical stifling. While baffles or other flow-directing devices can be used, they are not essential to this invention. In general, the average residence time is between about 50 and 1000, say 100 and 300, seconds.

The rate at which carbon monoxide and hydrogen can be consumed will be affected by the nature of the microorganism, the concentration of the microorganism in the aqueous fermentation broth and the fermentation conditions. As the rate of transfer of carbon monoxide and hydrogen to the aqueous fermentation broth is a parameter for operation, conditions affecting the rate of transfer such as interfacial surface area between the gas and liquid phases and driving forces are important.

A portion of the aqueous fermentation broth is withdrawn from time to time or continuously from the bioreactor for product recovery. Product recovery can consist of known equipment arrangements for removal of residual cell material, separation and recovery of liquid products from the fermentation liquid, return of recovered fermentation liquid and purging of waste streams and materials. Suitable equipment arrangements can include filters, centrifuges, cyclones, distillation columns, membrane systems and other separation equipment. U.S. Pat. No. 8,211,679 shows an arrangement for a product recovery bioreactor that recovers an ethanol product from a bioreactor.

Deep, Bioreactors. MLD Tank Reactors and Their Operation

Most forms of fermentation zones (also called bioreactors) are suitable for the bioconversion of syngas use with the invention. The chief requirements of bioreactors include: axenicity; anaerobic conditions; suitable conditions for maintenance of temperature, pressure, and pH; sufficient quantities of substrates, and nutrients are supplied to the culture; optimum mass transfer performance to supply the gases to the fermentation medium; and the end products of the fermentation can be readily recovered from the bacterial broth.

Types of fermentation apparatuses or bioreactors that are known to those of skill in the art have been disclosed elsewhere in this disclosure. Such bioreactors may be used alone or in combination with multiple bioreactors of the same or different types in series or parallel flow. These apparatuses will be used to develop and maintain the microorganism cultures used to produce the Liquid Products of this invention.

The fermentation reactors used in this invention may be of any suitable design; however, preferably the design and operation provides for a high conversion of carbon monoxide and hydrogen to oxygenated organic compound.

Fermentation reactors include, but are not limited to, bubble column reactors; jet loop reactors; stirred tank reactors; trickle bed reactors; biofilm reactors; high pressure bioreactor and static mixer reactors including, but not limited to, pipe reactors. Because of economy of capital cost and operation, deep tank bioreactors are preferred. Regardless of the type of deep tank bioreactor, especially where using microbubbles that promote a stable dispersion of bubbles in the aqueous broth, mixing currents exist that not only assure the relatively uniform aqueous phase composition but also increase the contact time between the gas bubbles and the aqueous broth.

Deep, MLD tank reactors are of a sufficient volume that the fermentation process is commercially viable. The height and aspect ratios of the commercial bioreactors are as characterized previously herein. While the reactors are typically circular in cross-section, other cross-sectional configurations can be used provided that uniformity in the liquid phase is obtained. The height of the aqueous menstruum will establish a hydrostatic pressure gradient along the axis of the reactor.

The deep, MLD tank reactors use one or more mechanical stirrers or impellers. Usually two or more impellers are used at different heights with higher aspect ratio reactors. The design of impellers for stirred tank reactors and their positioning within the reactors are well within the skill of a stirred tank reactor designer. Axial flow impellers are frequently used. Preferably the design of the impellers and the positioning of the impellers within the reactor take into consideration energy costs in rotating the impellers to obtain uniformity of the aqueous menstruum in the reactor.

The mechanical stirring should be sufficient to promote the uniformity of liquid composition through the reactor and need not, and preferably is not, used as a generator of a significant fraction of the microbubbles. The rotational speed of an impeller will depend upon the type of impeller, the number of impellers used, the configuration of the reactor and the volume of the aqueous menstruum. In general, the rotational rates are less than about 200 revolutions per minute, and often in the range of about 5 to 150 rpm. Despite the large volume of commercial-scale reactors, the speed at the tips of the impellers would not be so high as to cause undue damage to the microorganisms, yet the desired uniformity of liquid phase throughout the reactor can be readily achieved. The deep, MLD tank reactor may contain baffles or other static flow directing devices.

The depth of the aqueous menstruum in the deep bioreactors such as an MLD tank reactor will occupy either the full height or nearly the full height of the reactor. The height of the aqueous menstruum will establish a hydrostatic pressure gradient along the reactor. The dispersion of gas and liquid in the dispersion stream must overcome this hydrostatic pressure at the point where it enters the reactor. Thus if the gas feed enters at a point of 10 meters below the liquid surface the static pressure head inside the vessel would equal approximately 100 kPa gauge and for a liquid height of 15 meters the static pressure head would equal approximately 150 kPa gauge.

The conditions of fermentation, including the density of microorganisms, aqueous menstruum composition, aqueous menstruum depth and syngas residence time, are preferably sufficient to achieve the sought conversion efficiency of hydrogen and carbon monoxide which will vary depending upon the design of the fermentation reactor and its operation. The pressure may be subatmospheric, atmospheric or super atmospheric, and is usually in the range of from about 90 to 1000 KPa absolute and in some instances higher pressures may be desirable for biofilm fermentation bioreactors. As most bioreactor designs, especially for commercial scale operations, provide for a significant height of fermentation broth for the fermentation, the pressure will vary within the fermentation bioreactor based upon the static head.

Any suitable procedure may be used to start-up a deep, MLD tank reactor. Typically, the reactor is filled with a gas not containing reactive oxygen. Although a wide variety of gases for blanketing can be used, such as gases containing carbon dioxide, nitrogen or lower alkane, e.g., alkane of 1 to 3 carbon atoms such as methane and natural gas, cost and availability considerations play a role in the selection of the blanketing gas as well as its acceptability to the anaerobic fermentation process and subsequent unit operations. The reactor is partially charged with aqueous menstruum containing microorganisms and gas feed is provided to grow the culture of microorganisms and additional aqueous menstruum is provided until the aqueous menstruum has obtained the desired height in the reactor and the density of microorganisms has reached its desired level.

As stated above, a combination of bubble size and duration of contact with the aqueous fermentation broth are necessary to achieve these high conversions. For commercial operations, the fermentation operation preferably provides a total molar conversion of hydrogen and carbon monoxide in the substrate gas feed in the range of at least about 90, preferably at least about 95, mole percent. If required to provide adequate contact time between the gas bubbles and the aqueous fermentation broth more than one bioreactor may be used in gas flow series in the bioreactor assembly. The use of sequential, deep tank bubble column bioreactors is disclosed in U.S. Pat. No. 8,771,999. Use of a single stage deep tank fermenter that can achieve this same conversion efficiency and deep tank start-up procedures are disclosed in U.S. Pat. No. 8,936,927

A convenient control system for operating the processes of this invention involves determining the flow rate of the off-gas and analyzing the off-gas composition for carbon monoxide and hydrogen for comparison with the rate of carbon monoxide and hydrogen being provided by the fresh syngas to determine conversion efficiencies. The analysis may be conducted by any suitable method including but not limited to gas chromatography, mass spectroscopy, and infrared absorption as is well known. The rate of fresh syngas can be adjusted to achieve the targeted total conversion efficiency. Once steady-state operating conditions are achieved, the rate of off-gas recycle is typically maintained constant within the constraints of equipment limitations and so long as a major upset of the viability of the culture of microorganisms does not occur.

Gas Feed Supply and Injection:

The processes of this invention supply gas feed by injection using a motive fluid. Variations in the motive liquid flow rate can be used to modulate the microbubble size and thus modulate the rate of transfer of carbon monoxide and hydrogen to the liquid phase. The processes of this invention use at least one injector using a motive fluid for supplying gas feed to the aqueous menstruum. Preferably the reactor contains 2 or more injectors, and commercial scale reactors will often contain at least 2, often 4 to 8 or 10, laterals of injectors with as many as 100 or more injectors. The number of injectors used is typically selected based upon the ability to be able to transfer adequate amounts of gas substrate under steady-state operating conditions and to enhance cross-sectional uniformity of the gas phase in the reactor.

The injectors may be jet mixers/aerators or slot injectors. Slot injectors are preferred, one form of which is disclosed in U.S. Pat. No. 4,162,970. These injectors operate using a motive liquid. The injectors, especially slot injectors, are capable of operating over a wide range of liquid and gas flow rates and thus are capable of significant turn down in gas transfer capability while still obtaining suitable microbubbles. Thus complex shutdown and start-up of injectors can be minimized, if not avoided, under steady-state operations.

The injectors are characterized as having nozzles of at least about 1, often about 1.5 to 5, say, 2 to 4, centimeters as the cross-sectional dimension in the case of jet injectors or as the smaller cross-sectional dimension in the case of slot injectors. The large cross-sectional dimension of the injectors provides several benefits in addition to being able to produce microbubbles. First, they are not prone to fouling including where aqueous menstruum is used as the motive liquid as would be a sparger designed to produce microbubbles. Second, where the aqueous menstruum is used as the motive fluid, high momentum impact of the microorganisms with solid surfaces is minimized thereby minimizing the risk of damage to the microorganisms. Third, the energy required to provide microbubbles of a given size is often less than that required to form microbubbles of that size using a microbubble sparger. Fourth, a high turn down ratio can be achieved. And fifth, the microbubble size can be easily varied over a wide range.

The bubble size generated by the injectors will be influenced by, among other factors, the rate of liquid flow through the injector and the ratio of gas phase to liquid phase passing through the injector as well as characteristics of the aqueous menstruum itself including, but not limited to its static liquid depth. Consequently, an injector can be operated to provide a selected bubble size which enhances the ability to use the injector in a modulation mode, i.e., provide the adjustment in the rate of transfer of carbon monoxide to the liquid phase based upon the size of the colony and its ability of the colony to bioconvert the carbon monoxide. The modulation can be obtained by changing one or more of (i) the gas to liquid flow ratio to the injector thus changing the volume of gas feed and (ii) changing the rate of motive liquid and thus the bubble size which affects the rate of transfer of carbon monoxide from the gas phase to liquid phase. Additionally, modulation can be obtained by changing the gas feed composition and thus the mole fraction of carbon monoxide in the gas feed.

Preferably the gas feed is introduced by the injector into the menstruum in the form of microbubbles having diameters in the range of 0.01 to 0.5, preferably 0.02 to 0.3 millimeter. At a given flow rate of gas feed having a given composition to a reactor, the rate of transfer of carbon monoxide and hydrogen can vary widely depending upon the size of the microbubbles, the pressure and the design of the fermentation reactor and its operation. At start-up and where desired, larger bubble sizes, in the range of 100 to 5000 microns in diameter may be used. Also a portion of the gas feed may be introduced by sparging to generate large bubbles, say, 1 to 5 or 10, millimeters in diameter, for assisting in mixing the aqueous menstruum. The gas substrate may be introduced into the bottom portion of the deep, bubble column reactor as a gas stream or as a gas in liquid dispersion as disclosed in U.S. patent application Ser. No. 12/826,991, filed Jun. 30, 2010. The presence of the oxygenated organic compound and/or other surface active agent enhances the formation of fine microbubbles.

The flow rate of motive liquid used in an injector will depend upon the type, size and configuration of the injector and the sought bubble size of the gas feed. In general, the velocity of the dispersion stream leaving the injector is frequently in the range of 0.5 to 5 meters per second and the ratio of gas to motive liquid is in the range of from about 1:1 to 3:1 actual cubic meters per cubic meter of motive liquid.

The microbubbles form a stable gas-in-water dispersion. The introduction of the microbubbles into the aqueous menstruum places the microbubbles in a dynamic environment. The height of the aqueous menstruum means that microbubbles in the dispersion will experience different static pressure heads as they travel upwardly through the reactor. Increased pressure will, with all else substantially the same, reduce the size of a microbubble. For a given gas feed rate, a greater surface area will be provided by the smaller microbubbles which enhances mass transfer. The size of a microbubble will also be affected by the diffusion of gases from the microbubble to the liquid phase. As carbon monoxide and hydrogen constitute a significant mole fraction of the microbubble as it is introduced into the aqueous menstruum, the dynamic conditions will promote a population of microbubbles that have small diameters to aid in maintaining the gas-in-water dispersion throughout the reactor.

The injectors may be located at one or more locations in the reactor and oriented in any suitable direction. Often the injectors are oriented to promote admixing of the gas feed with the aqueous menstruum and distribution in the reactor. The injectors may be located in a lower portion of the deep, MLD tank reactor. However, an advantage provided by using a deep, MLD tank reactor is that injectors may be placed at two or more heights. Due to the mechanical mixing, the dispersion introduced will be relatively uniform throughout the reactor and the average gas residence time will be advantageous to assure the sought transfer of carbon monoxide and hydrogen to the liquid phase. By locating the injectors over the height of the reactor, the uniformity of composition of the gas-in-liquid dispersion in the aqueous menstruum is promoted and less mechanical stirring energy may be required to maintain the sought uniformity.

Adverse Component Removal

In some instances, the bioreactor used for the syngas bioconversion may be used for the bioconversion that removes the Adverse Component. In other instances, a separate bioreactor The Adverse Component that is contained in the aqueous fermentation broth for the syngas bioconversion can be from any source, including, but not limited to, an impurity in the feed gas and a metabolic product during the fermentation by the microorganism provided to produce the product oxygenated organic compound or by an adventitious microorganism contained in the fermentation broth.

The removal of the Adverse Component may be prompted by one or more events. One such event is an unplanned event. One type of unplanned event is where normal procedures to avoid the presence of, or limit the concentration of, the Adverse Component in the fermentation broth fail. Another type of unplanned event is where the fermentation broth contains an unexpected microorganism that produces the undesired organic compound. Another type of event is a sporadic or intermittent event such as an anticipated build-up of the Adverse Component which is an inherent co-product.

The Adverse Component can be in a concentration such that the microorganism population for the bioconversion of syngas is substantially killed or is materially, adversely affected, either or both by inhibition and cell death. Alternatively, even if the Adverse Component does not adversely affect the microorganisms for the bioconversion of syngas, its build-up can adversely affect operation. For instance, the build-up may affect the ability to maintain a steady-state operation of a continuous process for making the product oxygenated organic compound.

The time of implementation of the processes of this invention thus can vary according to the cause and effect of the Adverse Component. Thus, the processes may only be used when an unplanned event occurs that adversely affects the population of the microorganisms for bioconverting syngas. Alternatively, the processes may be used intermittently or sporadically to address a build-up of the Adverse Component. This mode of implementation can be beneficial in those instances where higher concentrations of the Adverse Component can have an effect on the metabolic pathways used by the microorganism for the bioconversion of syngas which leads to an accelerated production of the Adverse Component.

The processes of this invention can be used in the presence of viable microorganisms used for the bioconversion of syngas. However, the sustenance of the population of these microorganisms may be problematic in the event that syngas feed is disrupted. Some microorganisms for the conversion of syngas may also bioconvert nitrate anion, but the product of the bioconversion may be ammonium cation rather than nitrogen. In some instances, aqueous fermentation broth has a substantial absence of microorganisms for the bioconversion of syngas. For instance, the microorganisms for the bioconversion of syngas are removed from the aqueous fermentation broth or the broth is denatured prior to being contacted with the denitrifying microorganisms.

The aqueous fermentation broth which is to be treated in accordance with the processes of this invention may contain the product oxygenated organic compound. The pH of the aqueous fermentation broth being treated is preferably greater than about 5.0, and is preferably at least about 5.5. Since the denitrifying microorganisms can metabolize the product oxygenated organic compound, preferably the product oxygenated organic compound is recovered from the fermentation broth prior to implementing the processes of this invention. Nevertheless, there may be instances where the concentration of the product oxygenated organic compound in the fermentation broth is so low that its recovery may not be practical. Frequently, where the product oxygenated organic compound is recovered, the mole ratio of the product oxygenated organic compound to the Adverse Component in the fermentation broth is less than about 1:1, say, less than about 0.25:1, and sometimes less than about 0.1:1. Where the product oxygenated organic compound is ethanol and the Adverse Component is acetic acid, distillation can reduce this ratio to about 0.01:1 or less.

The processes of this invention can be used in respect of processes for bioconverting syngas to any product oxygenated organic using an aqueous fermentation broth. The preferred product oxygenated organic compounds are alcohols. Where the product oxygenated organic compound is alcohol, typically carboxylic acids are co-produced and, in some instances, these carboxylic acids comprise the Adverse Component. In some instances, other alcohols can also be an Adverse Component, e.g., n-butanol in the production of ethanol or propanol where n-butanol is toxic at a lower concentration to the microorganisms.

For continuous operations, the residence time of the aqueous fermentation broth in the bioreactor should also be sufficient to achieve the sought reduction in the undesired organic compound.

Denitrification

In one aspect of this invention nitrate anion may be added by the process of this invention to treat the broth. In the metabolic process of denitrifying, oxygenated organic compound is metabolized, i.e., the nitrate is reduced and the organic compound is oxidized. For instance, where acetic acid is the undesired organic compound, the following represents the overall metabolic reaction:

2Na⁺+2NO₃⁻+1.25CH₃COO⁻+1.25H⁺→N₂+20H⁻+2.5CO₂+2Na⁺+1.5H₂O.

The nitrate anion may be supplied in any convenient form to the fermentation broth which is treated in accordance with this invention. Typically the nitrate anion is supplied as a solid to be dissolved in the aqueous fermentation broth or as a concentrated aqueous solution of dissolved nitrate anion. The amount of nitrate anion required for the denitrification to effect the desired reduction of the undesired organic compound in the aqueous broth may be added initially, or may be added continuously or intermittently as the denitrification proceeds. Often, the peak concentration of nitrate anion is less than about 5, preferably less than about 1, grams per liter of the aqueous fermentation broth, and most often is between about 50 and 1000 milligrams per liter. The lower concentrations of nitrate anion tend to provide a treated fermentation broth that contains little, if any, nitrite anion. Accordingly, the nitrate reduction products will be normally gaseous such as molecular nitrogen, nitrous oxide and nitric oxide, and are exhausted as off-gas.

The amount of nitrate anion added will depend upon the total oxidizable organic content of the organic carbon compounds in the aqueous fermentation broth, and the sought reduction of the Adverse Component. Those skilled in the art of denitrification are readily capable of determining the amounts of nitrate anion required to achieve the sought reduction of undesired organic compound. In some instances, the processes of this invention will be operated to achieve a certain carbon oxygen demand level in the fermentation broth. In these instances, the COD of the treated aqueous fermentation broth is frequently less than about 5000, preferably less than about 1000, and in some instances less than about 500, milligrams of oxygen per liter.

Often a water-soluble salt or acid of the nitrate anion or mixture thereof is used as the source of nitrate anion. Suitable nitrate salts include, but are not limited to, ammonium, alkali metal (preferably one or more of sodium, potassium and cesium), and alkaline earth (preferably calcium) nitrate. As discussed below, the use of nitrate salts results in the pH of the aqueous medium increasing as the denitrification proceeds. Once a sought pH is obtained, nitric acid may be used as all or a portion of the nitrate anion to maintain a sought pH.

The denitrifying microorganism can be naturally occurring (wild-type) or recombinant, including, but not limited to, genetically modified. As ample types of denitrifying microorganisms are naturally-occurring, these are preferred due to availability, robustness and mitigating any risk of microbial contamination if released to the environment. Examples of denitrifying bacteria include, but are not limited to, Thiobacillus denitrificans, Micrococcus denitrificans, Paracoccus denitrificans, Spirilum, Cornebacteriaum, Cytophata, Alcaligenes and Pseudomonas.

The denitrifying microorganisms can be maintained in a bioreactor until needed or can be stored as a freeze-dried or concentrated liquid culture. Thus the processes utilizing this aspect of the invention can be started-up in a short period of time even from an inoculum to obtain a population of denitrifying microorganisms desired to achieve a sought density of denitrifying activity per unit volume of fermentation broth being treated.

The denitrifying microorganism can be in a free suspension in the aqueous fermentation broth or may be in the form of a supported biofilm or otherwise immobilized or encapsulated in a solid structure. Support materials may be selected from any hydrophobic or hydrophilic material including, but not limited to, bone char, diatomaceous earth, zeolite, ceramics, clay, wood chips, glass, sand, activated carbon and polymeric materials such as polyethylene, polypropylene, polystyrene, polysulfones, polyacrylates, polymethacrylates, polyesters, nylons, cellulosics, and polyvinyls such as polyvinyl alcohol and polyvinyl chloride. The denitrifying microorganisms may be supplied as an inoculum at the time the process for removing the undesired organic compound needs to be used, e.g., where the denitrifying microorganisms are in a free suspension in the aqueous fermentation broth or are grown on a support during the denitrification process. Alternatively, a prepared solid structure already containing the denitrifying microorganisms on or entrapped or immobilized in the solid structure can be used. Such solid structures are available from Hitachi, Japan; Ecomat, Hayward, Calif.; Lentikats, Czech Republic; and Microvi Biotech, Hayward, Calif.

Many denitrifying microorganisms are mesophilic and thus are operable within the temperature range used for the bioconversion of syngas. In these instances, the temperature of the aqueous fermentation broth is within the range of about 25° C. to about 40° C. The head pressure during the denitrification is not critical to the broad aspects of this invention, but usually is in the range of between about 50 and 1000 kPa.

Moreover, since the denitrification products are normally gaseous and evolve from the aqueous fermentation broth as an off gas, the denitrification does not itself lead to any unduly adverse component being generated in the aqueous fermentation broth.

Where the treated aqueous fermentation broth contains denitrifying microorganisms, it is usually the case that the microorganisms are removed from the treated aqueous fermentation broth or the broth denatured such that substantially no viable denitrifying microorganisms are present when the syngas bioconversion is commenced. However, with denitrifying microorganisms that are incapable of surviving under the conditions of the syngas bioconversion, e.g., are not able to tolerate the acidic conditions of the syngas bioconversion or are incapable of surviving in the presence of syngas, removal of the microorganisms may not be essential. The use of such denitrifying microorganisms is particularly useful where the denitrification is conducted in the bioreactor in which the treated aqueous fermentation broth is to be used for the bioconversion of syngas. In some aspects of this invention where the denitrifying microorganisms are not capable of escaping from an immobilizing solid, a unit operation to remove the microorganisms or to denature the broth may not be required.

Where the pH of the aqueous fermentation broth has been allowed to increase during the denitrification, it may be useful to lower the pH prior to reusing the aqueous fermentation broth to convert syngas. Lowering the pH can be accomplished in any suitable manner. Preferably, the concentration of nitrate anion in the treated aqueous fermentation broth is below that which could adversely affect the microorganisms used for the bioconversion of syngas prior to introducing these microorganisms into the broth. Often the nitrate concentration in the treated fermentation broth is less than about 10, and sometimes less than about 5, parts per million by mass per liter of broth. However, some microorganisms for the bioconversion syngas to oxygenated organic compound are able to tolerate presence of nitrate anion, or any adverse effect caused by the nitrate anion, is reversible.

The reduction of nitrate salts results in an increase alkalinity of the aqueous fermentation broth. Hence, initially the pH may be below a desired range for the denitrification, but over time the pH of the aqueous broth will reach that desired range. Nitric acid can be used as all or a portion of the nitrate anion supplied if the pH becomes higher than desired.

Adjuvants and nutrients, including micronutrients, may be added to fermentation broth undergoing denitrification. Since the aqueous fermentation broth is from the bioconversion of syngas, at least some nutrients, including micronutrients, typically will already be contained in the broth.

Renewable Feedstock from Waste Material

The high moisture content, renewable feedstock can be preconditioned to remove contaminants, reduce the particle size and/or thermally preprocessed to increase the rate and/or degree of degradation of the organics in the feedstock streams. The high moisture, renewable feedstock may also be pretreated to concentrate the organic content by removing water therefrom before entry into the anaerobic digester if desired. Mesophilic (˜35° C.), thermophilic (˜55° C.) or phased (use of both thermophilic and mesophilic steps in sequence) operating temperatures are both suitable for use in the anaerobic digestion processing aspects of this invention. The high moisture feedstock material stream can be processed alone or preferably in conjunction with processing of excess or waste biosolids generated from the Syngas fermentation process itself.

Use Biogas

The biogas produced includes multiple components or fractions which are readily useable in the overall plant. Uses of the various fractions are described below.

Methane in the biogas may be used as a supplemental energy source in any device requiring a gaseous energy source such as steam boilers, thermal oxidizers, and burners or Steam Methane Reformers (SMR), as examples. The biogas may also be reformed to produce additional syngas. Although others have advocated that biogas can be reformed using any number of catalytic based reforming technologies, (such as Steam Methane Reformers [SMR] or Auto Thermal Reformers [ATR]) including U.S. Pat. No. 8,187,568 and Offerman U.S. Pat. No. 8,198,058, this is expensive since the H₂S (and other reduced sulfur compounds in the biogas) and siloxanes present due to the partial degradation of silicone based compounds that are present in many high moisture feedstock's, require extensive and expensive pretreatment to protect the precious metal based catalysts. Therefore the preferred reforming technologies are non-catalytic partial oxidation (NC-POX) or plasma arc-type gasification (either can use air or oxygen) since no pretreatment is needed and in fact the H₂S, useful as a sulfur source in the syngas fermentation, is, at least in part, preserved and present in the syngas produced and can be fed directly to the fermentation process where it can be used as a source of cell S. Other similar operational schemes to those listed above are possible by those skilled in the art. If a NC-POX or plasma gasifier is used to reform the biogas, the resultant syngas will have a relatively low e⁻/C ratio that usually does not exceed 4.0 and more often is less than 3.5, which indicates that not all the carbon oxides (CO₂ and CO) will be converted to the liquid products produced in the syngas fermentation processes of this invention if just this syngas stream is used. This affords an opportunity to blend this gas with a second syngas that has a high e−/C that is usually at least 7.0 and more often at least 7.6 such as SMR reformed NG or certain waste industrial gases that tend to have high H₂ concentrations. The result is a high fraction of the carbon in both feedstocks (or combined syngas) can be converted to the soluble organic carbon compounds produced—an overall very effective carbon capture process. The electron to carbon ratio of the blended substrate gas will have an e⁻/C ratio in the range of about 5.4:1 to 6.7:1, preferably between about 5.6:1 to 6.5:1, and most preferably between about 5.8:1 to 6.3:1

By another aspect of the instant invention, an organic waste stream and excess biosolids (recovered, in part, from the Syngas fermentation) are co-digested in a conventional CSTR type anaerobic digestion (AD) system. The biogas produced in this case is not reformed but is forwarded to the primary Syngas fermentation reactor. It can be compressed and blended with the feed Syngas, or if fermenter tail gas recycle is employed, added to that stream on a low pressure side of a compressor or other such device used to recycle the gas. It can also be independently added at a point higher up in the fermenter where the need for CO₂ is the greatest. This allows the CO₂ in the biogas to be used to reduce the e⁻/C ratio into the preferred range in cases where the feed gas has relatively high e⁻/C ratio.

The CH₄ in the biogas is essentially inert in the Syngas fermentation process and passes with the tail gas which can be recovered and used for the energy value within the plant.

Sulfur Supply

As mentioned, the reduced sulfur is used as an essential nutrient in anaerobic Syngas fermentation so the H₂S can help offset the need for sulfur addition. The level of H₂S can be manipulated upward, if desired, by adding additional oxidized sulfur from sources, such as those disclosed in this application and in the form of SO₄, to the primary anaerobic digestion system as well. Use of high oxidation state sulfur is described in U.S. patent application Ser. No. 13/546,703 filed Jul. 11, 2012 the contents of which are hereby incorporated in their entirety

In other aspects processes of this invention use calcium sulfite to provide at least a portion of the sulfur needs for the microorganisms used for the bioconversion of syngas. Calcium sulfite is recognized as a food additive, particularly as a food preservative, and as an antimicrobial agent. The low solubility of calcium sulfate in combination with the sulfur requirement need of the microorganisms enables an effective supply of sulfur nutrient as all or a portion of the sulfur nutrient requirement under normal operation or during an interruption in the supply of a different sulfur nutrient normally used in the syngas bioconversion process. The concentration of dissolved calcium sulfite in the aqueous menstruum is often in the range of about 20 to 50 milligrams per liter.

In accordance with this aspect of the processes of this invention solid calcium sulfite is maintained in the aqueous menstruum. The calcium sulfite can be provided (direct addition or in situ) continuously or intermittently to the aqueous menstruum in amounts sufficient to provide undissolved solids of calcium sulfite. The continuous or intermittent addition may occur substantially over the period of time that the bioconversion process is operating either to provide substantially all or a portion of the sulfur nutrient or as a reservoir in the event of a disruption of the supply of a different sulfur nutrient normally used. Alternatively, calcium sulfite can be used only in the event of a disruption in the feed of a normal sulfur nutrient source, the provision of calcium sulfite may commence when the disruption occurs.

The rate of the provision of calcium sulfite can be determined by any suitable parameters. Analytical techniques exist for a determination of the concentration of calcium sulfite solids in the aqueous menstruum; however, they tend to be time consuming. For instance, an aliquot fraction of the aqueous menstruum can be filtered using a 0.2 micron filter to collect solids and the presence of solid calcium sulfite ascertained by X-ray crystal diffraction. Accordingly and most conveniently, the rate of provision of calcium sulfite is based upon the expected metabolic rate of consumption of sulfur by the population of microorganisms in the aqueous menstruum. Also the sulfite concentration in the aqueous menstruum can be monitored and if it is below saturation, the rate of provision of calcium sulfite is increased. In an indirect method, the hydrogen sulfide concentration in the off-gas from the aqueous menstruum can be monitored. If the concentration changes without change in the population density of the microorganisms, the rate of provision of calcium sulfite can be altered to provide a hydrogen sulfide concentration within a targeted range based on the metabolic activity of the microorganism.

Recovery of Nutrient Values

Through degradation processes occurring during anaerobic digestion, a significant fraction of the nitrogen (N) and phosphorus (P) organically bound in the organic materials being digested is transformed into recoverable inorganic, bioavailable forms useable in the fermentation process, primarily NH₄⁺/NH₃ and ortho PO₄. It is also possible to concurrently recover a significant fraction of the water needed for the fermentation with the recovered nutrients, if desired. The digestate stream may also contain other potentially valuable nutrients such as vitamins, amino acids, etc.

In one exemplary embodiment, the digestate stream is first sent to a liquid/solids separation unit, such as a centrifuge or screw press, and the centrate/pressate is then further processed to recover the N, P, and potentially, water, in a useable form. In one exemplary embodiment, this involves processing the centrate stream from a solids separation using a filtration process that includes a 1 to 10, preferably 1 to 5 kilodalton (kD) ultrafiltration (UF) membrane. The membrane system retains most of the larger Molecular Weight (MW) dissolved proteins, sugars and the like, whose build-up could adversely affect the syngas fermentation process, as a small volume retentate stream while providing a UF filtrate stream that contains most of the value of the digestate in a form that can be readily made suitable for addition to the fermenter. Additional treatment to remove remaining “contaminants” such as using granular activated carbon (GAC) adsorption and/or oxidation processes and/or thermal processing can be used to further clean and/or sterilize the UF filtrate prior to its use, if desired.

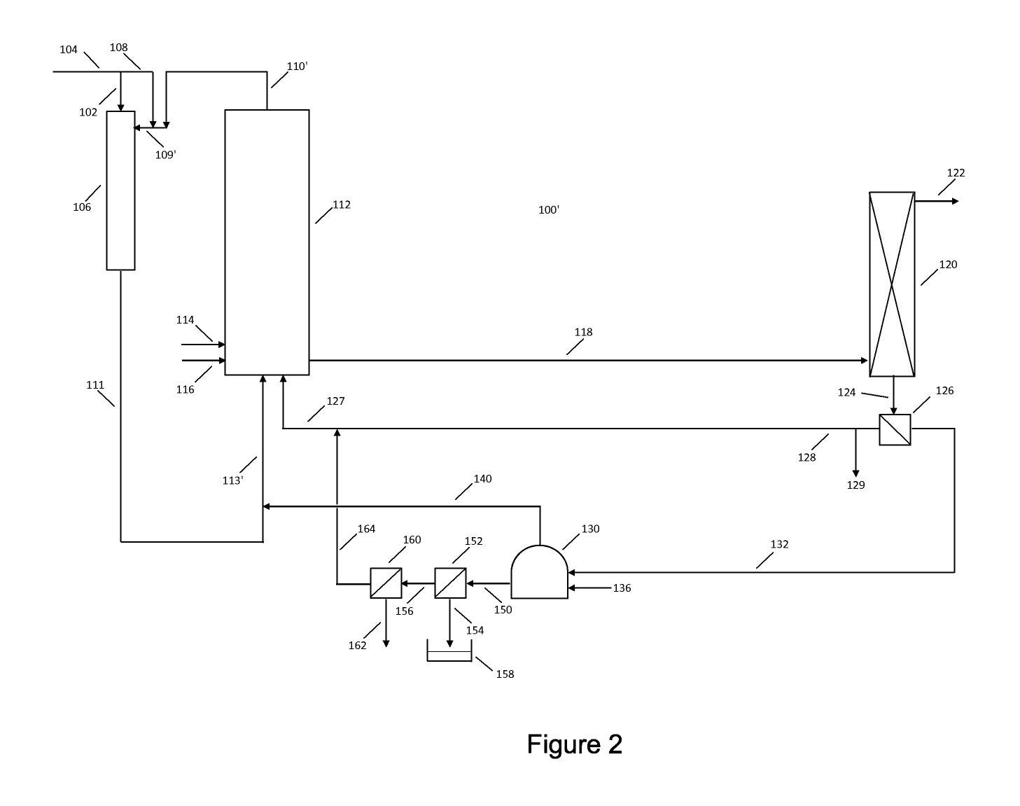

What is shown in FIG. 2 is the centrate being processed in such a membrane system where the water, N and P are carried through as a permeate stream resulting in an N and P rich stream containing only very limited amounts of undesirable dissolved organics. Other undesirable compounds such as large proteins, sugars, etc., captured in the retentate can be sent either to waste water treatment (WWT), or, if sufficiently concentrated, is sent to the AD system to recover the energy value. For example, this stream may also be sufficiently concentrated thorough evaporation to high total solids (TS) “syrup” which may be directly disposed of with the dewatered AD digestate solids, for example.

Other approaches to recovering these nutrients are possible, such as stripping/condensing or flashing ammonia from the AD centrate/pres sate stream and/or precipitating the PO4 in a form that can be subsequently recovered and then further processed so it can be used in the syngas fermentation, are also contemplated.

The processing aspects as illustrated would also allow recovery of approximately up to 80 to 90% of the water from the AD centrate/pressate as the permeate stream, which, in water shortage areas, could be extremely valuable. In addition, later portions of the text detail the amount of savings related to the recovery of the N, P, and S as well as the potential value of the generated CH₄ and CO₂.

Recovery of Trace metals

The dewatered AD digestate solids stream is generally between 18% and 25% total solids. This “cake” solids stream can be disposed of in a number of ways (land application, landfilling, etc.,) or it can be processed in a manner that allows recovery of the trace metals. To do this, the cake solids following dewatering are then dried to ˜90% to 92% TS and sent on to a gasifier. The syngas produced in the gasifier can be either added to the syngas “substrate” going to the fermentation processing steps used to supply a significant fraction of the energy needed for drying the biosolids or used elsewhere in the plant for energy and/or heat. Waste heat from the overall syngas to chemicals process can be used to supply some or all of the heat required for drying which in many cases allows better use of the syngas produced.

It is possible to co-digest material that enhances the dewaterability of the AD digestate (due to additional fiber for example) and or increase the energy content of the digestate solids. This reduces the drying requirements and/or increases the net energy recovered during the gasification step.

The ash that is produced during gasification has a mass that is on the order of 3% to 5% of the Solids sent to gasification (on a dry weight basis) and can be landfilled, or land applied (perhaps qualify as biochar). This ash is, however, rich in phosphorus (P) and all the trace metals that are added to the fermentation process and taken up by the cells. In the syngas fermentation itself, citric acid is used as a chelant to prevent precipitation and ensure the bioavailability of certain required trace metals. Addition of citric acid (or other acceptable chelating agent) to the ash (that contains these metals) to achieve a pH of ˜3 to 4 is a sufficiently low pH to “leach” most of the metals and remaining P from the ash into the aqueous phase as a concentrated solution. This solution is then separated from the ash and cleaned as necessary for use back in the fermentation.

### DETAILED DESCRIPTION OF THE PREFERRED EMBODIMENTS

Referring now to the Figures in greater detail, there is illustrated therein a schematic representation of the various aspects of the process for carrying out the methodology of the invention in a variety of different forms. The illustration of the various aspects is not presented as limiting but merely as exemplary.

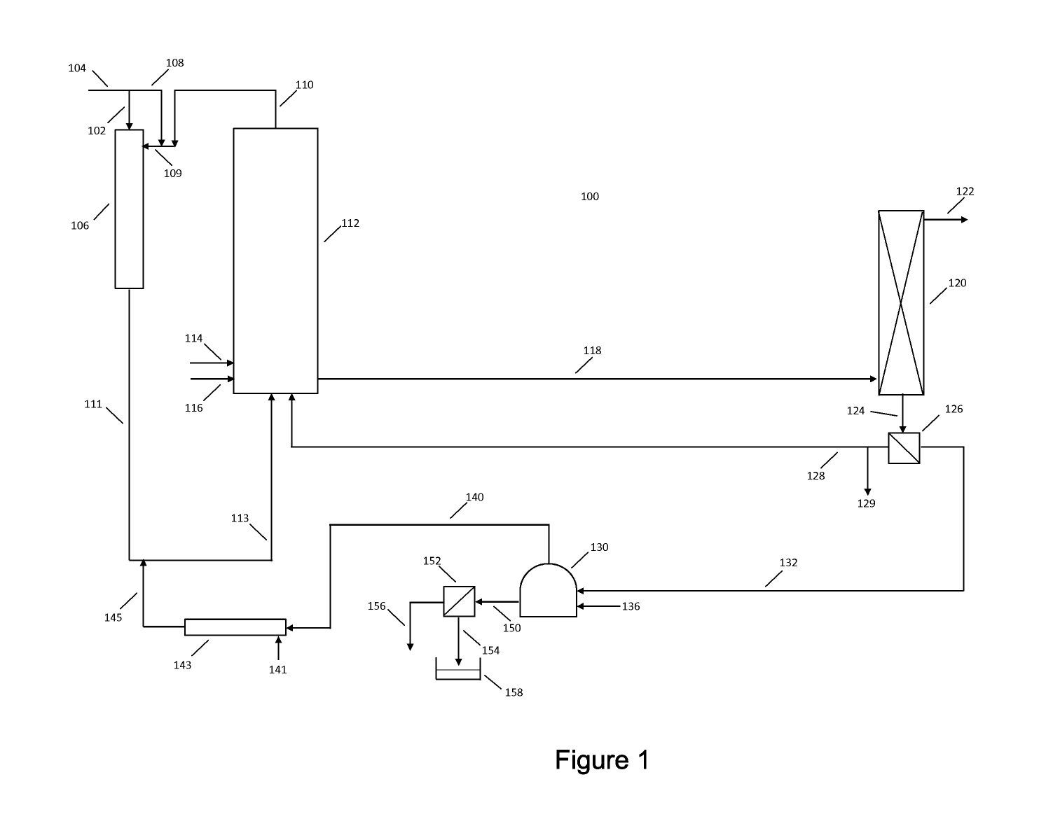

FIG. 1

FIG. 1 shows the portions of the process needed to when practicing the aspect of anaerobically treating a high moisture, renewable feedstock material in an anaerobic digestion (AD) process to produce a biogas primarily containing CH₄, CO₂ and smaller amounts of H₂S. The anaerobic digestion process can be accomplished using conventional CSTR, plug flow digesters, a dry (or solid state) anaerobic digester (generally with moisture levels of between 50% and 80%) or any number of other configurations such as two-stage and two phase digestion, a combination of a dry fermentation system with liquid recycle through a high rate anaerobic treatment reactor (such as an upflow anaerobic sludge bed (UASB) or higher rate variants such as the IC (Internal Circulation) or EGSB (Expanded Granule Sludge Bed), anaerobic fluidized bed reactor (AnFBR) or an anaerobic filter (AF)), as examples.

FIG. 1 shows one arrangement for the needed process elements when including the anaerobic digestion aspect o the invention wherein NG from a suitable source (104) is delivered via a line 102 to reformer tubes of a steam methane reformer (SMR) 106 and where the NG is converted to a Syngas with a high e⁻/C ratio. A portion of the NG from the source is also provided via a line 108 to fuel burners (not shown) in the reformer 106, together with tail gas from the fermenter via line 110, to supply all the energy needed for the burners via line 109. The Syngas formed in reformer 106 is fed via line 111 and 113 to fermenter 112 into which are also fed water via line 114 and nutrients via line 116 which are necessary for viability, reproduction and performance of the microorganisms within the fermenter 112, which microorganisms act on the Syngas to transform it into ethanol, in the disclosed embodiment. Any suitable microorganism can be utilized in the fermenter 112, as there are known a plurality of such which produce ethanol or others may be used to provide corresponding other desired products, as noted above.

A fermenter broth exiting fermenter 112 and comprising, in this instance, ethanol, water, and an amount of suspended biological solids, exits the fermenter via line 118 and is fed into a distillation tower 120. The ethanol is separated from water/solids contained in the product depleted broth and collected overhead from column 120 via line 122 for dehydration. The suspended solids and water, referred to as still bottoms, is fed via a line 124 to a suitable separator 126 for separation of solids from the liquid portion of the stream or still bottoms. The liquid portion of the stream, with solids removed therefrom, is referred to as clarified still bottoms, and is returned via line 128 to the fermenter 112 for further reprocessing. A slip stream of clarified bottoms can be sent to wastewater treatment either continuously or intermittently via line 129 to prevent the build-up of dissolved salts and/or biological metabolites to concentrations that can become inhibitory to the fermentation process. The solids containing stream separated from the clarified still bottoms, is carried via line 132 into any suitable anaerobic digester 130 where the solids are digested to produce biogas containing predominantly CH₄ and CO₂. Additional high moisture, renewable feed stock stream(s) can be also be added to anaerobic digester 130 via line 136 to increase the amount of biogas produced and nutrients available for recovery and subsequent use back in the syngas fermentation processes. Suitable sources for the high moisture, renewable feed stock stream may be provided in the form of food wastes, such as from cafes, cafeterias, grocery stores, hotels, homes, etc., in a preferred embodiment.

Digestion of the contents of the streams carried by lines 132 and 136 to anaerobic digester 132 will yield two streams, one of which is a CH₄ and CO₂ containing stream carried by line 140 to a non-catalytic partial oxidation reformer 143, which, is concurrently fed a source of oxygen via line 141. The biogas and oxygen containing stream are reformed via non-catalytic partial oxidation reactor 143 into a syngas with a low e⁻/C ratio that is carried away via line 145. This syngas stream is then blended with the high e⁻/C ratio syngas in line 111 to produce a syngas with the optimal e⁻/C ratio to allow high conversion efficiencies of both the H₂/CO₂ and CO in the syngas, and sent to fermenter 112 in line 113. A liquid digestate stream also exits the anaerobic digester via line 150 and is carried to a solids separation unit 152 which separates and concentrates the digested solids which are removed via line 154 to storage tank 158 for transport off-site for various uses, such as land application for fertilization of soil, landfilling etc. The water stream from separation unit 152 is carried via a line 156 to a waste water treatment facility (not shown) for processing.

FIG. 2

In a modified form from that of FIG. 1 the process of the invention may incorporate the aspect of anaerobically treating a high moisture, renewable feedstock material in an anaerobic digestion (AD) process by incorporating elements as shown in FIG. 2, for which the description of FIG. 1 holds with the following modifications. In this operating scenario the biogas produced in digester 130 is carried via line 140 and blended with reformed NG gas stream 111 where the added CO₂ from the biogas is sufficient to generate a combined stream (line 113′) that has an optimal e⁻/C ratio to allow high conversion of the H₂/CO₂ and CO in fermenter 112. The tail gas that exits fermenter 112 via line 110′ has not only the unreacted H₂ and CO but all the CH₄ from the biogas in line 140. This stream is blended with sufficient NG from line 108 to produce a combined feed to the reformer hot box as line 109′ that has sufficient energy to operate the hot box of the reformer.

With regard to the water stream exiting the solids separation unit 152 via line 156, the water stream is taken by line 156 and fed through a UF filtration unit 160 that has a molecular weight cut off of between 1 to 5 KD. UF unit 160 retains most of the proteins and sugars as retentate stream 162 that is removed and sent to a waste water treatment facility. The cleaned, ultra-filtered water stream, or nutrient rich uncontaminated filtrate which is carried by line 164 can be blended with the flow in line 128 for delivery to the fermenter 112 as combined stream 127. The ultrafiltration system also removes contaminants such as bacteria which could adversely affect the microorganisms in fermenter 112. Note a pretreatment step prior to treatment of stream 156 in UF unit 160 may be needed in some cases (not shown).

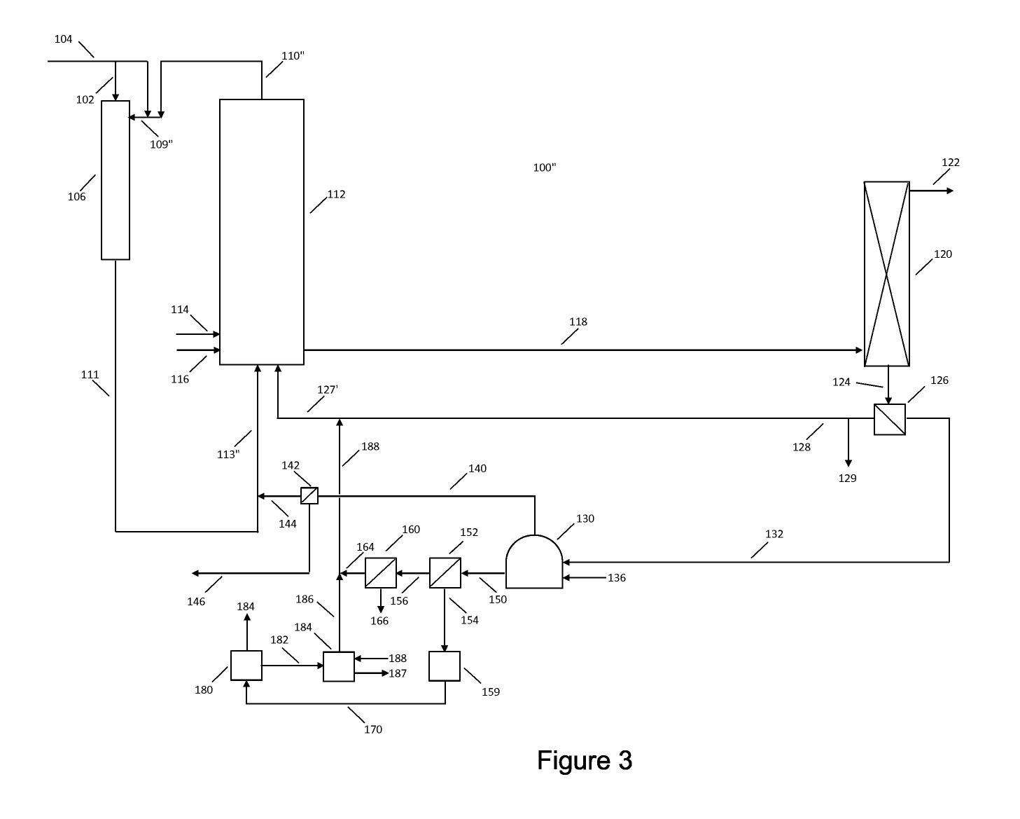

FIG. 3

FIG. 3 has the same features as FIG. 2 with the following exceptions. The biogas in line 140 (from AD unit 130) is passed to a gas separation unit 142 that captures the CO₂ and H₂S which is then is taken in line 144 and blended with syngas feed in line 111 to generate a combined stream 113″ that has an e−/C ratio that allows high conversion efficiency of the H₂/CO₂ and CO in the syngas fed to fermenter 112. Any conventional CO₂ capture process, such as water scrubbing, solvent scrubbing (amines, selexol, etc.) or membrane separation processes can be used to capture the CO₂.

The reject stream 146, is primarily CH₄ that can be used for energy in the process. Use in the SMR would require removal of the siloxanes to a low level so is not a preferred use.

Additionally, in this figure the dewatered solids from digester 130 contained inline 154 are sent to dryer 159 where they are converted to stream 170 that has on the order of 8% to 10% moisture remaining. The solids in 170 are then fed to biosolids gasifier 180 that generates a syngas stream 190, that can be used for energy in the plant, used to dry the biosolids or even cleaned and used as feed to fermenter 112. The ash generated in gasifier 180 is carried away inline 182 to leach reactor 184. Citric acid is added via line 188 to 184 either continuously or batch-wise at sufficient amounts that a concentrated citric acid stream, rich in the trace metals and P, is leached from the ash and is recovered as stream 186. Stream 186 is blended with stream 164 forming stream 188 that is mixed with stream 128 and recycled back to fermenter 112 via line 127′. The remaining “leached” ash is removed for disposal via line 187.

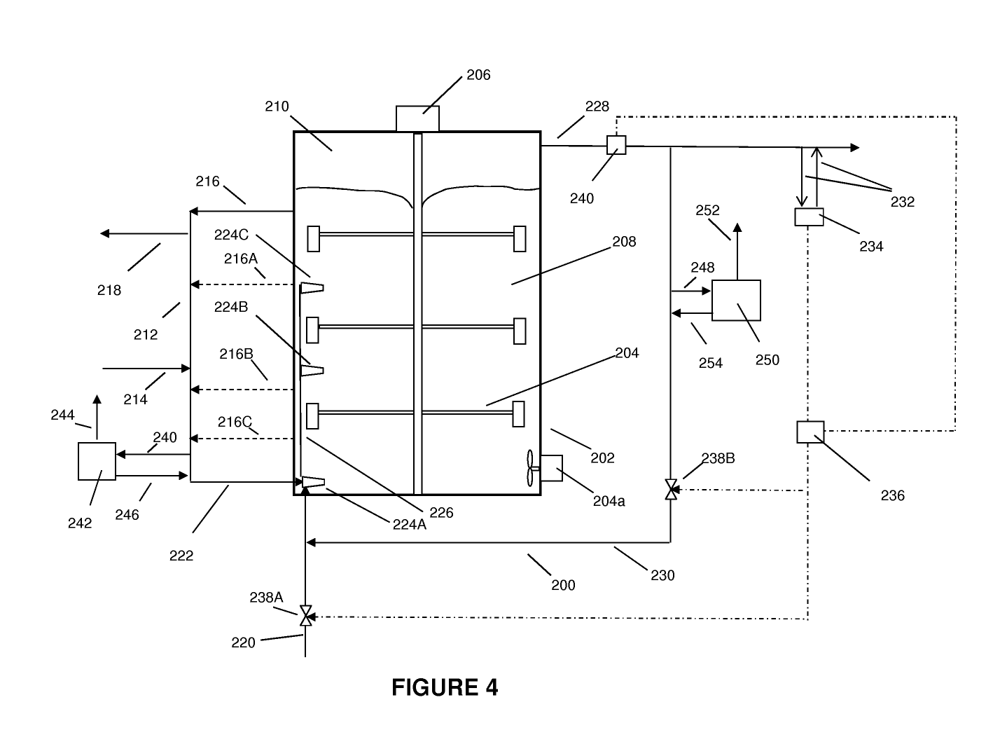

FIG. 4

FIG. 4 shows the continuous stirred tank fermentor aspect of this invention as an assembly 200 comprising tank 202 having therein agitator 204. Agitator is shown as having three stirring blades on a center shaft; however, fewer or more blades can be used. Motor 206 powers the agitator and controls the revolutions per minute. An aqueous menstruum 108 is contained in tank 202. Above aqueous menstruum 208 in tank 202 is overhead space 210.

Aqueous menstruum is withdrawn from tank 202 via line 216 for product recovery and for recycle. As shown, line 216 is adapted to withdraw from the upper portion of liquid menstruum 208. The fermentor assembly 200 is adapted to operate at less than its full liquid capacity, e.g., during start-up operations. Lines 216A, 216B and 216C are provided to enable aqueous menstruum to be withdrawn from upper portions of lower volumes of aqueous menstruum. Each of lines 216, 216A, 216B and 216C are adapted to be in fluid flow communication with liquid header 212. Liquid header 212 is in fluid communication with line 218 for withdrawal of a portion of the aqueous menstruum for product recovery and purge. Line 214 provides make-up liquid to header 212. The make-up liquid provided by line 214 may be one or more of broth from a seed farm, recycle liquid from product recovery, and make-up water.

Syngas is provided to fermentor assembly 200 via line 220. The syngas is introduced in admixture with recycled off-gas as will be described below as a gas feed into aqueous menstruum 208 in the form of microbubbles. To achieve the microbubbles, gas feed and motive liquid, which is obtained from liquid header 212 and supplied by line 222, are passed to nozzles 224A, 224B and 224C. The motive liquid and gas feed are passed via line 226 to the other nozzles. Each nozzle may be a jet nozzle, or preferably, a slot injector. In a commercial scale unit more nozzles would be employed.

Off-gas from overhead zone 210 is removed via line 228. A portion of the removed off-gas may be treated to remove oxygenated organic compound and exhausted. Another portion of the removed gas is recycled to tank 202 via line 230.

The off-gas is analyzed to determine carbon monoxide and hydrogen compositions. As shown, gas analyzer 234 is located in communication with line 228 via lines 232 to withdraw and return gas samples. Analyzer 234, for purposes of this depiction is a gas chromatograph/mass spec. Analyzer 234 is in data communication with control processor 236 which is a computer containing algorithms to determine the conversion efficiency of carbon monoxide and hydrogen in the fresh syngas. Control processor 236 is also in data communication with flow meter 240 which is adapted to determine the off-gas flow rate from tank 202. Control processor 236 is in data communication with valve 238A in line 220 to adjust the rate of fresh syngas supply to obtain the targeted conversion efficiency. In the event that the analysis indicates that the rate of recycle needs to be adjusted, control processor 236 is also in data communication with valve 238B.

Assembly 200 is provided with a unit operation to remove carbon dioxide from the aqueous menstruum. As shown, recycling aqueous menstruum in header 212 is withdrawn via line 240 and passed to flash tank 242. Flash tank 242 is maintained under a lower pressure, usually about ambient atmospheric pressure, and thus carbon dioxide effervesces and is removed via line 244. The aqueous menstruum with a reduced carbon dioxide content is returned to header 212 via line 246.

Carbon dioxide can also be removed from the recycling off-gas. As shown, recycling off-gas is withdrawn from line 230 via line 248 and passes to carbon dioxide removal unit operation 250. Carbon dioxide removal unit operation 250 may be any suitable device. For instance, carbon dioxide can be removed by sorption into an aqueous stream containing ethanol and the sorbent then regenerated to yield carbon dioxide which is removed via line 252. The recycling off-gas with a reduced carbon dioxide concentration is returned to line 230 via line 254.

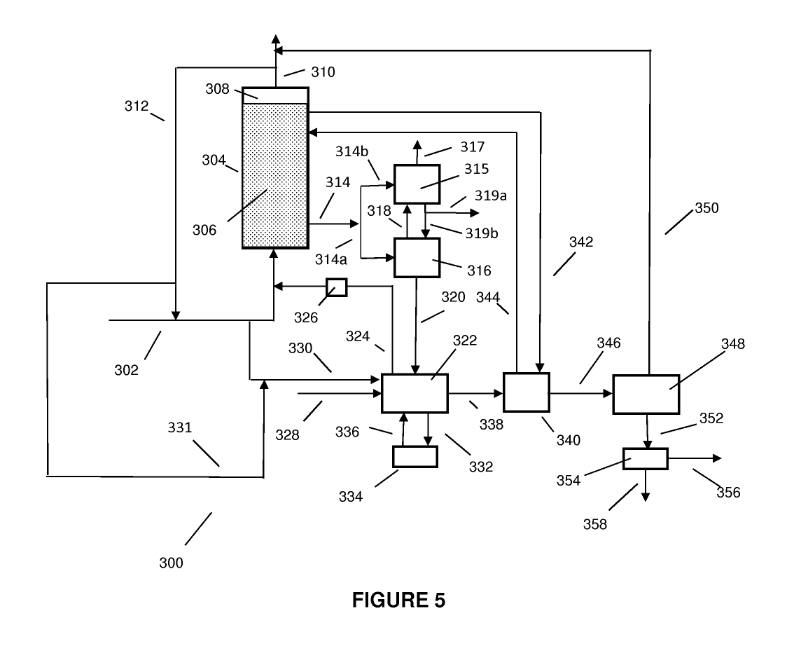

FIG. 5

FIG. 5 shows the necessary process elements for the integration of sulfur supply into the process of this invention. In this aspect syngas is provided to apparatus 300 via line 302.

Syngas in line 302 is passed to reactor 304 containing fermentation broth 306. Fermentation broth is maintained under fermentation conditions and the syngas is provided therein in a manner to enhance mass transfer of hydrogen and carbon monoxide to the aqueous broth for bioconversion by microorganisms to oxygenated organic compound. The fermentation may be on a continuous or batch basis. Preferably the syngas is continuously supplied.

As shown, reactor 304 has head space 308 containing off-gas which is unreacted hydrogen, carbon dioxide and carbon monoxide, and inerts such as methane and nitrogen. Off-gas is withdrawn via line 310. A portion of the off-gas, if desired, can be recycled via line 312 to increase the conversion of syngas to product.

Intermittently or continuously an aliquot portion of the fermentation broth 306 is withdrawn via line 314. Where the fermentation is a batch fermentation, essentially all the fermentation broth would be removed at one time. The portion withdrawn in a continuous operation is sufficient to maintain the oxygenated organic compound concentration in the fermentation broth below that which unduly adversely affects the microorganisms.

As shown, all or a portion of the withdrawn fermentation broth can be directly passed via line 314a to separator 316 which may be a decanter, filter, centrifuge or hydrocyclone to provide an aqueous liquid phase containing oxygenated organic compound and having a substantial absence of solids and a solids-containing phase which is usually a slurry, e.g., from between about 25 to 90, mass percent solids (excluding water contained in the solids). The aqueous liquid phase is passed via line 318 to product recovery operations 315 which can comprise one or more of distillation, membrane separators, and the like. For purposes of this description, product recovery operations 315 shall be referred to as distillation assembly 315. Alternatively, or in addition, all or a portion of the withdrawn fermentation broth can be directed via line 314b to distillation assembly 315.

Distillation assembly 315 comprises one or more distillation columns and a still bottoms separator. Ethanol is recovered via line 317. If a solids-containing portion of the fermentation broth is provided via line 314b to distillation assembly 315, then a solids-containing phase, which contains dead cells (due to the temperature conditions of the still) and solid proteins is withdrawn via line 319b and sent to separator 316. Otherwise the bottoms fraction is removed via line 319a.

The solids-containing phase is passed from separator 316 via line 320 to anaerobic digester 322. If desired, a portion of the solids-containing phase can be returned to reactor 304 by a suitable line (not shown). If so, the portion returned should enable an average cell retention to be maintained at a desired level to provide a balance between productivity and cell growth and rejuvenation rates.

Anaerobic digester 322 is maintained under anaerobic conditions for the sought catabolic activity. Any suitable microorganism for the digestion of biomass can be used. In one preferred embodiment of the invention, anaerobic digester 322 is maintained under acidogenic digestion conditions. Microorganisms for bioconversion of biomass to carboxylic acids such as formic, acetic, propionic, butyric and lactic acids under anaerobic conditions are well known. Often the anaerobic digester is self-inoculated. The conditions for the anaerobic digestion can vary depending upon the microorganisms used. Preferably the pH is maintained at or below about 6 such that free hydrogen sulfide is favored. digester 322 may be of any suitable design and is usually a stirred tank reactor.

Where anaerobic digester 322 is operated under methanogenic conditions, the residence time in the digester is usually sufficient to achieve the sought degradation of the solids to provide a solids mass that can be sent to disposal. Where anaerobic digester is an acidogenic digester and is to be followed by a methanogenic digester, the operator may elect to maintain the residence time sufficient to achieve a desired recovery of hydrogen sulfide or a desired production of carboxylic acid.

Anaerobic digester produces a biogas which is withdrawn via line 324. The biogas composition will depend upon the nature of the anaerobic digestion. For conventional anaerobic digestion, the biogas will frequently contain about 50 to 70 volume percent methane, about 25 to 45 volume percent carbon dioxide with the balance being primarily water vapor and hydrogen sulfide. Acidogenic digestion generally provides a biogas relatively free of methane which contains 40 to 90 volume percent carbon dioxide with the balance being hydrogen, water vapor and hydrogen sulfide.

Biogas in line 324 is passed through device 126 to remove any carry over microorganisms and is directed to reactor 304. Device 326 can be a filter or any other method that allows the gas stream to remove microorganisms, and preferably viruses, or otherwise be sterilized prior to going to the syngas fermentation processing steps of this invention. The advantage of the invention is that the biogas is not treated to remove hydrogen sulfide. Moreover, with the hydrogen sulfide being dilute in the biogas, handling and safety risks are reduced. Even though the hydrogen sulfide is being provided in a dilute form, often containing between about 500 and 100,000 ppmv hydrogen sulfide, the low molar flow rate of the biogas, often less than about 2, and most often less than about 1, percent of the molar flow rate of the syngas feed, there is no appreciable adverse effect on the syngas fermentation.

Anaerobic digester 322 may additionally be used to bioconvert added sulfoxy moieties and elemental sulfur to hydrogen sulfide. Line 328 provides sulfur or sulfur compounds to be reduced to hydrogen sulfide to anaerobic digester 322. As stated before, sulfuric and sulfurous acids are preferred and aid in maintaining a desired pH. The amount of sulfur moiety provided is preferably such that the biogas from anaerobic digester contains the sought amount of hydrogen sulfide to meet the requirements of the microorganisms in reactor 304. The amount to be provided can be calculated or may be in response to measurements. For instance, the hydrogen sulfide content of the off-gases can be determined and the amount of sulfur moiety provided increased or decreased to maintain the concentration in the off gases within a predetermined range. Often the amount of hydrogen sulfide required to be supplied to a reactor to meet nutrient needs of the microorganisms is in the range of 0.5 to 1.0% of the total cell mass grown in the fermenter.

The bioconversion of the sulfoxy moiety to hydrogen sulfide requires an electron donor. In most instances sufficient the electron donor exists in the anaerobic digester, e.g., from the biomass from the syngas fermentation. If additional electron donor is required, a suitable source of electron donor is the syngas. Conveniently a portion of the syngas may be passed to anaerobic digester 322 from line 302 via line 330. The amount of syngas required will depend in part upon the composition of the syngas and the amount of donor needed. As the syngas will be combined with the biogas for passage to reactor 304, the use of an excess amount of syngas can be used. Generally, about 1 to 10, say, about 2 to 5, volume percent of the syngas may be passed to anaerobic digester 322. The syngas provided by line 330 may also be used to sweep hydrogen sulfide from the anaerobic digestion liquor. In addition or alternatively, sweep gas may be provided by the recycling off-gas from line 312 passed to anaerobic digester 322 via line 331.

Cell disruption reactor 334 may be used to break open the cells, such as the Molecular Chemical Grinder technology offered by PMC Bio Tec, LLC, of Exton, Pa., and thereby enhance the rate of digestion of the solids. As shown, solids-containing liquid is withdrawn from anaerobic digester 322 via line 332, and subsequent to treatment is returned via line 336.

Where only anaerobic digester 322 is used, the solids-containing effluent from the digester can be directed to solids dewatering unit operation 354 which provides an aqueous effluent via line 356 for waste water treatment. A dewatered solids product is withdrawn via line 358 for solids disposal.

As depicted, the solids-containing effluent from anaerobic digester 322, which for purposes of the following description is an acidogenic digester, is passed via line 338 to electrodialysis reversal unit 340 for recovery of carboxylic acids. See, for instance, Electrodialysis (ED) and Electrodialysis Reversal (EDR), U.S. Department of the Interior, Bureau of Reclamation, Sep. 20, 2010. Other separation unit operations include, but are not limited to, electrodialysis, ion exchange membranes, ultrafiltration and liquid-liquid extraction. Fermentation broth is passed via line 342 from reactor 304 to ion exchange column 340 where carboxylic acid is recovered from the ion exchange resin and is returned via line 344 to reactor 304. Where ethanol is the sought oxygenated organic compound, the carboxylic acids are metabolized by microorganisms in the fermentation broth to generate additional ethanol and thus increase the overall conversion efficiency of syngas to ethanol.

The solids-containing effluent is then passed via line 346 from ion exchange column 340 to anaerobic digester 348 which is a methanogenic digester. Anaerobic digester 348 is maintained under methanogenic conditions. Any suitable microorganism for bioconversion of biomass to methane under anaerobic conditions may be used and frequently the anaerobic digestion liquor is self-inoculating. The conditions for the anaerobic digestion can vary depending upon the microorganisms used. Anaerobic digester 322 may be of any suitable design including, but not limited to, bubble column reactors; jet loop reactors; and stirred tank reactors.

The methanogenic conditions in anaerobic digester 348 provide a methane-containing biogas and a slurry of digested solids. The methane-containing biogas will frequently contain about 50 to 70 volume percent methane and about 25 to 45 volume percent carbon dioxide with the balance being primarily water vapor. Often the hydrogen sulfide concentration is less than about 10 ppmv, preferably less than about 1 or 2 ppmv. The biogas is withdrawn from anaerobic digester 348 via line 350 and can be used for any suitable purpose, usually without further treatment to reduce sulfur content. As shown, the biogas is combined with the off-gas from reactor 304. The combined gases, which due to the combination with the methane-containing biogas, will have a slightly higher energy density. This gas may be thermally oxidized to provide heat, e.g., to dry biomass for gasification to generate syngas.

The slurry of digested solids is removed from anaerobic digester 348 via line 352 to be sent to dewatering unit operation 354. Water is removed from dewatering operation via line 356 and sent to waste water treatment. Dewatered solid are removed via line 358 for solids disposal.

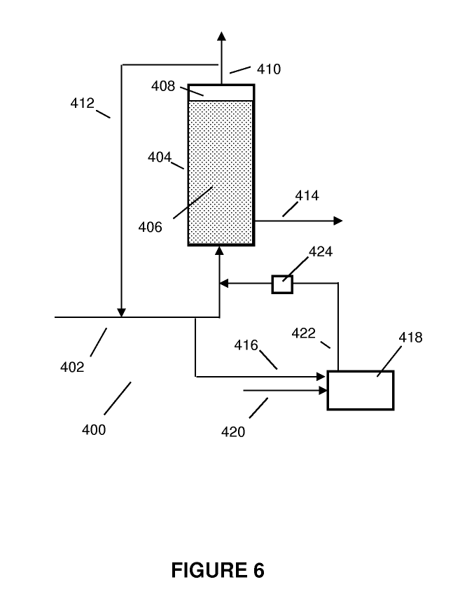

FIG. 6

With respect to FIG. 6, the process arrangement 400 as shown therein provides hydrogen sulfide as a nutrient to a fermentation broth for converting syngas to oxygenated organic compound. The syngas in line 402 is passed to reactor 404 containing fermentation broth 406. Fermentation broth is maintained under fermentation conditions and the syngas is provided therein in a manner to enhance mass transfer of hydrogen and carbon monoxide to the aqueous broth for bioconversion by microorganisms to oxygenated organic compound. Off-gas is withdrawn from head space 408 via line 410. A portion of the off-gas, if desired, can be recycled via line 412 to increase the conversion of syngas to product. Intermittently or continuously an aliquot portion of the fermentation broth 406 is withdrawn via line 414 for product recovery.

As shown, a portion of the syngas is provided via line 416 to sulfoxy reactor 418. Alternatively, a portion of the off-gas form the syngas fermenter may be used. Also provided to sulfoxy reactor 418 is sulfoxy moiety via line 420. For purposes of illustration only, the sulfoxy moiety is sulfuric acid. Sulfoxy reactor 418 contains microorganism for the bioconversion of sulfate to hydrogen sulfide. Biogas is withdrawn from sulfoxy reactor 418 via line 422 and is passed through filter 424 and then to reactor 404.

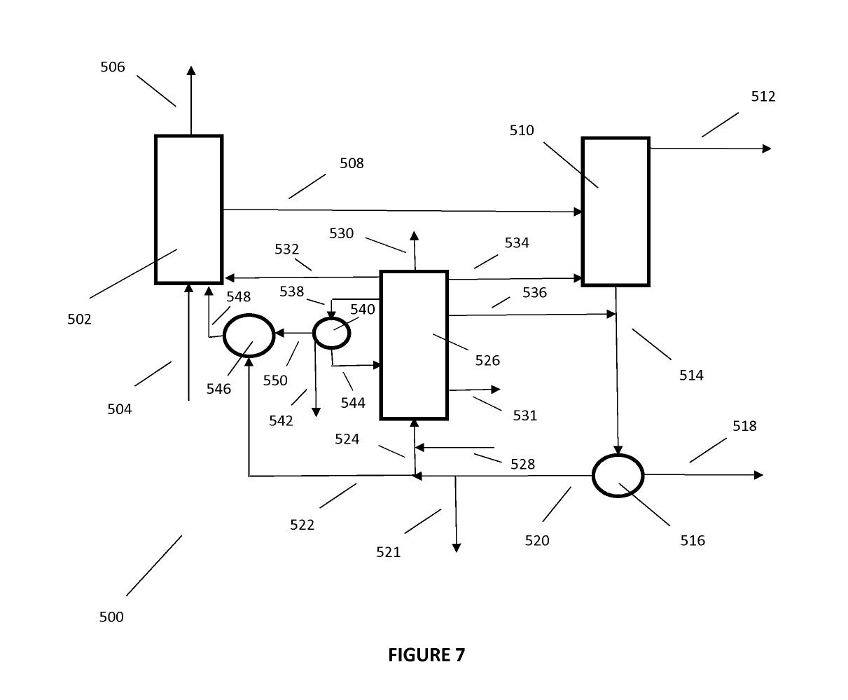

FIG. 7

FIG. 7 is a schematic depiction of an apparatus generally designated as 500 suitable for practicing processes in accordance with this invention The description of FIG. 7 describes the Adverse Component removal aspect of this invention in the context of the recovery and production of ethanol, however this aspect of the process is readily adaptable to making other oxygenated products such as acetic acid, butanol, propanol and acetone.

FIG. 7 shows a bottoms stream containing solids from the microorganisms and proteins precipitated from solution in an aqueous phase is passed via line 514 to a solids separation unit operation 516, which for purposes of discussion is a centrifuge. The bottoms stream also contains higher boiling organic compounds such as acetates. A solids-rich stream is removed from centrifuge 516 via line 518, and the solids-rich stream can be processed for waste recovery, e.g., in an anaerobic digester. Centrifuge 516 also provides an aqueous stream which exits via line 520 and can be redirected to fermentation reactor 502 via line 522. As shown, a portion of the aqueous stream in line 522 can be withdrawn as an aqueous purge via line 521. Generally, the rate of aqueous purge is at least sufficient to maintain a desired, steady-state ionic balance in the aqueous fermentation broth in fermentation reactor 502. The aqueous purge can be directed to a waste water treatment unit operation.

Line 522 is shown as directing the broth to sterilizing unit operation 546 which for purposes of discussion is a steam heated tank to increase the temperature of the fermentation broth sufficiently to effect sterilization. The sterilized broth is cooled to the desired temperature and returned to fermentation reactor 502 via line 548. Where the temperature of the distillation assembly is sufficient to denature the bottoms stream and the denitrifying microorganisms do not survive under the conditions in the fermentation reactor, sterilizer 546 need not be used.

Line 520 from centrifuge 516 is also capable of directing the aqueous stream to line 524 to be passed to denitrification reactor 526. As shown, nitrate anion (salt and/or nitric acid) can be provided via line 528 to denitrification reactor 526. Denitrification reactor 526 contains denitrifying microorganisms that are either maintained in the reactor as a normal course, when the invention is practiced in continuous mode, or they can be inoculated via line 529 when operated in a batch mode. Off-gases generated by the denitrification exit denitrification reactor 526 via line 530. The batch mode, which is used when a fermentation has reached the point that the adverse compound has reached a level requiring a complete restart of the syngas fermentation, is described first.

In batch mode operation where a restart of the syngas fermentation is required, the broth from fermenter 502 is sent to distillation assembly 510 with the bottoms stream being directed by line 514 to solids separation unit 516. The aqueous stream from solids separation unit 516, which contains a low concentration of solids is passed via line 524 to denitrification reactor 526 and the aqueous stream is accumulated and concurrently treated by adding nitrate through line 528 and a denitrifying inoculum through line 529. Once fermenter 502 is sufficiently empty the treated fermentation broth can be withdrawn from denitrification reactor 526 to refill fermenter 502.

The Figure shows several options. The treated fermentation broth can be (i) directly returned to fermenter 502 through line 532 or (ii) sent to solids separation unit 540 via line 538 to remove solids or (iii) can have a portion directed to each path. The solids-containing stream (which contains viable denitrifying microorganisms) can be sent back to reactor 526 via line 544 or passed to a waste treatment facility via line 542. The fermentation broth from which solids have been removed can be passed from solids separation unit 540 via line 550, line 560 and line 548 to fermenter 502 for reuse as the fermentation broth.

In the continuous or semi-continuous mode, the supply rates of both the aqueous stream in line 524, which contains a carbon source for the denitrifying organisms such as an organic acid, and nitrate anion from line 528 are such that the population of denitrifying microorganisms can be maintained. To maintain steady-state operation, a portion of the aqueous fermentation broth in denitrification reactor 526 is removed via line 532. The Figure depicts three options for handling the removed broth. In one option, the removed portion of the fermentation broth is passed via line 534 to distillation assembly 510. The broth is denatured in distillation assembly 510. However, depending upon the volume of the broth removed, the heat load for distillation assembly 510 might be unacceptably increased. In a second option, the broth removed is passed via line 536 to line 514. Centrifuge 516 separates the solids and returns fermentation broth to denitrification reactor 526 and/or fermentation reactor 502 via lines 520 and 522. In the third option, the removed broth is passed to line 560 and then to fermenter 502 via line 548. In a fourth option, the removed broth is passed via line 538 to solids separation unit operation 540 (e.g., a centrifuge) to provide a solids-rich stream which exits via line 544 and a solids-depleted stream that can be sent back to fermentation reactor 502 via line 550, line 560 and then line 548. A portion of the solids-rich stream in line 544 returns denitrifying microorganisms to denitrifying reactor 526 via line 545, and the remaining portion is purged via line 542.

When denitrification reactor 526 is used to continuously or intermittently reduce Adverse Component concentration contained in the aqueous stream being returned to fermentation reactor 502, a number of options exist. The process of this invention may be used to prevent an undue build-up of the Adverse Component. In this option, fermentation reactor 502 can continue to operate in a normal mode to produce ethanol. In another option, the syngas feed to fermentation reactor 502 is ceased and the volume of aqueous fermentation broth is decreased or fully removed. The withdrawn fermentation broth is passed to distillation assembly 510 to recover ethanol and is passed to denitrification reactor 526 to bioconvert organic compound. Holding tanks, not depicted, can be used to hold the treated fermentation broth until the desired reduction of volume of fermentation broth has been removed from fermentation reactor 502. In a third option, the syngas feed to fermentation reactor 502 is ceased but the volume of fermentation broth in fermentation reactor 502 is not reduced as treated fermentation broth from fermentation reactor is returned to fermentation reactor 502. Initially, the fermentation broth from fermentation reactor 502 is directed to distillation assembly 510. When the concentration of ethanol in fermentation reactor 502 becomes so diluted that it no longer is efficient to recover ethanol, the fermentation broth withdrawn via line 508 can be directed to line 514 or to denitrification bioreactor 526.

An advantage of the continuous mode operation includes the ability to use denitrification reactor 526 to reduce the load on the wastewater treatment facility in that at least a portion of the liquid purge can be the treated fermentation broth. As shown, line 531 is adapted to remove treated fermentation broth from line 532 for purging. Thus, the volume of fermentation broth passed to denitrification reactor 526 can vary from the rate of the liquid purge to that rate required to effect both the liquid purge and the reduction of Adverse Component.

### EXAMPLES

### Example 1

This calculated example relates to the aspect of the invention that involves the addition of source separated organics (SSO), particularly food wastes that have been treated to remove contaminants and macerated or pulped to generate a slurry suitable for co-digesting in an AD along with excess biosolids produced during the syngas fermentation. The resulting biogas stream is reformed using a non-catalytic partial oxidation reformer. This is in turn then blended with SMR reformed NG to produce a combined syngas with the preferred e−/C ratio to achieve a high conversion efficiency of both H2/CO2 and CO to soluble oxygenated products.

The SSO slurry is assumed to have a composition similar to that presented in Table 1, which is based on the characterization of a combined FW mixture from cafeterias, grocery markets and hotels (Zhang et al., 2007).

TABLE 1
Characterization of Food Wastes

| Parameter | Units | Concentration |
|---|---|---|
| TS | % | 30.9 |
| VS (volatile solids) | % | 26.3 |
| N | % dw | 3.16 |
| P | % dw | 0.52 |
| COD/VS | g/g | 1.55 |

This example assumes a nominal 12 million gallon/year (MGY) ethanol facility wherein 190 wet tons per day of raw SSO is processed, including removal of 5% of the total mass as contaminants, into a 10% TS slurry and co-digested with the excess biosolids from the fermentation process to produced biogas.

The composition of the NC-POX reformed biogas, the SMR reformed NG streams and the blend of the two syngas's is shown in Table 2. The NC-POX is fed with ˜650 scfm of CH4 along with 350 scfm of CO2. The SMR is fed with ˜1650 scfm of NG.

The POX is used as the reformer for the biogas to reduce the amount of compression and eliminate any cleanup prior to reforming (no need for H₂S or siloxanes removal prior to the POX—a considerable savings in capex and opex). This also preserves all of the carbon in the biogas and produces a syngas with a lower e⁻/C ratio that when blended with SMR reformed NG achieves the desired e⁻/C ratio for the fermentation process. A comparison of the composition of the two reformed gas streams and the blend that delivers the desired e⁻/C is presented in Table 2.

TABLE 2
Comparison of Different Syngas Compositions (mole fractions)

| | | | Blend of SMR |
|---|---|---|---|
| Parameter | SMR-NG | POX-Biogas | and POX |
| H2 | 0.752 | 0.451 | 0.691 |
| CO | 0.177 | 0.479 | 0.238 |
| CO2 | 0.062 | 0.070 | 0.063 |
| N2 | 0.004 | — | 0.003 |
| CH4 | 0.006 | — | 0.004 |
| e⁻/C Ratio | 7.77 | 3.39 | 6.17 |

Up to 80 gpm of water is recoverable with the UF membrane approach outlined previously. In such an approach there is an expected recovery of approximately 90% of the digestate stream as centrate from dewatering and that approximately 80% of the centrate stream can be recovered as UF permeate. The N, P and S requirements for the syngas fermentation processs are shown in Table 3 along with the mass that can reasonably be recovered within this embodiment of the invention.

TABLE 3
Mass of nutrients that AD followed by recovery of a centrate with a
tight (approximately 1 KD) UF membrane can provide for Syngas
in a nominal 12 MGY Syngas to Ethanol Plant

| | | Needed | | | Avail- | |
|---|---|---|---|---|---|---|
| | | for | Available | | able | Overall |
| | | Fermen- | from | Available | from | Percent |
| Nutrient | Units | tation | Biosolids | from SSO | Biogas | Recovered |
| Nitrogen | lb/d | 1,925 | 420 | 822 | | 95.8 |
| Phos- | lb/d | 162 | 52 | 85 | | 84.6 |
| phorus | | | | | | |
| Sulfur | lb/d | 130 | | | 60 | 46.2 |

As described above, the various aspects that may form part of the invention provide a number of advantages, some of which are described above and others of which are inherent in the invention. Further, modifications may be proposed to the process arrangements shown in FIGS. 1-7 and the other embodiments and aspects of the invention without departing from the teachings herein. Accordingly, the scope of the invention is only to be limited as necessitated by the accompanying claims.

1. A process for bioconverting CO, H2 and CO2 to oxygenated organic compound comprising:
a. passing a gas feed comprising CO, H2 and CO2 into a primary reactor containing aqueous fermentation broth under aerobic fermentation conditions, said fermentation broth containing microorganisms adapted for bioconverting the gas feed to oxygenated organic compound, to produce oxygenated organic compound dissolved in the fermentation broth and an off gas;
b. maintaining in said primary reactor a depth of fermentation broth of at least 10 meters;
c. maintaining in said reactor a head space above the upper portion of the fermentation broth;
d. continuously supplying the gas feed to said aqueous menstruum through an injector that uses a motive liquid to form a stable gas-in-liquid dispersion in the fermentation broth;
e. bioconverting carbon monoxide and hydrogen and carbon dioxide to an oxygenated organic compound and providing off-gas from the aqueous menstruum in the head space;
f. withdrawing from the head space of said reactor at least a portion of the off-gas;
g. removing from the syngas reactor at least an aliquot portion of the fermentation broth containing oxygenated organic compound and containing biosolids;
h. separating from said aliquot portion of the fermentation broth an aqueous biosolids-containing phase containing biosolids having a higher solids content and a reduced oxygenated organic compound concentration than said aliquot portion;
i. recovering at least a portion of the oxygenated organic compound;
j. recovering from the biosolids-containing phase an essential compound for the bioconversion of the gas feed to the oxygenated organic compound; and,
k. returning the essential compound to the aqueous fermentation broth in the primary reactor.

2. The process of claim 1 wherein the off-gas is mixed with the gas substrate in an amount sufficient to (i) achieve a conversion efficiency of the total moles of carbon monoxide and hydrogen in the gas substrate to oxygenated organic compound of at least about 80 percent and (ii) attenuate the risk of carbon monoxide inhibition of the microorganism used for the bioconversion.

3. The process of claim 1 wherein the biosolids-containing phase passes to anaerobic digestion at conditions to biodegrade solids in the aqueous liquid phase to provide an aqueous degraded solids product and a biogas product comprising hydrogen sulfide.

4. The process of claim 3 wherein a sulfur moiety comprising at least one of sulfoxy moiety and elemental sulfur is supplied to the anaerobic digestion step and at least a portion of the sulfur moiety is bioconverted in step to hydrogen sulfide.

5. The process of claim 4 wherein the anaerobic digestion conditions are acidogenic fermentation conditions.

6. The process of claim 5 wherein organic acid is produced in step (d) and is selectively removed and passed to the fermentation broth.

7. The process of claim 5 wherein after being subjected to acidogenic fermentation conditions, the aqueous degraded solids product is subjected to anaerobic, methanogenic digestion conditions to produce a methane-containing biogas and a further biodegraded, aqueous solids product.

8. The process of claim 4 wherein the sulfur moiety is supplied in an amount sufficient to maintain a predetermined range of hydrogen sulfide concentration in the off-gas.

9. The process of claim 6 wherein after being subjected to acidogenic fermentation conditions, the aqueous degraded solids product is subjected to anaerobic, methanogenic digestion conditions to produce a methane-containing biogas and a further biodegraded, aqueous solids product.

10. The process of claim 9 wherein at least a portion of the methane-containing biogas is directly or indirectly combined with the off-gas.

11. The process of claim 1 wherein the biosolids-containing phase passes to anaerobic digestion at conditions to biodegrade solids in the aqueous liquid phase to provide an aqueous degraded solids product and a biogas product that is combined with reformed natural gas (NG) and passed to the primary reactor.

12. The process of claim 11 wherein the biogas is reformed using a non-catalytic partial oxidation reformer to produce a reformed biogas with a first e⁻/C ratio and the reformed natural gas has a second e⁻/C that exceeds the first e⁻/C and the portion of at least one of reformed biogas and a reformed natural gas is combined with at least a portion of the gas feed in an amount to produce a combined syngas with a desired e⁻/C ratio.

13. The process of claim 1 wherein during the bioconveting the fermentation broth contains undissolved calcium sulfite.

14. The process of claim 13 wherein at least a portion of the undissolved calcium sulfite is an in situ precipitate The process of claim 2 wherein at least a portion of the undissolved calcium sulfite is an in situ precipitate occurring in the fermentation broth.

15. The process of claim 14 wherein the calcium sulfite is added to the fermentation broth as an aqueous slurry.

16. The process of claim 15 wherein the calcium sulfite added comprises calcium sulfite solids having a maximum particle size dimension of between about 1 and 100 microns.
